(19) United States
(12) Patent Application Publication
(10) Pub. No.: US 2006/0276089 A1
(43) Pub. Date: Dec. 7, 2006

(54) Cooling garment

(75) Inventors: Gandara Amarasinghe, Victoria (AU); Robert Shanks, Victoria (AU); Margaret Glewis, Victoria (AU); David Edward Mainwaring, Victoria (AU); Dorothy Forster, Victoria (AU)

Correspondence Address: DORSEY & WHITNEY LLP, 555 CALIFORNIA STREET, SUITE 1000, SUITE 1000, SAN FRANCISCO, CA, 94104, US

(73) Assignee: Royal Melbourne Institute of Technology, Melbourne (AU)

(21) Appl. No.: 10/565,319

(22) PCT Filed: Jul. 16, 2004
(86) PCT No.: PCT/AU04/00956

§ 371 Date: Aug. 11, 2006

(51) Int. Cl. A61F 7/00

(52) U.S. Cl. 442/121; 442/123; 442/286; 428/72; 607/108

(57) ABSTRACT

An article of clothing comprising a phase change material which is a blend of at least two compounds and which has a melting point of from 5 deg C. to 30 deg C., and a melting temperature range of from 1 to 5 deg. C.

The present invention relates to garments, and especially, clothing which is specifically adapted to provide thermoregulatory control. The garments in accordance with the invention provide active thermal control and in this respect are different from passive systems which function by means of insulation intended to retain body heat or to prevent adverse increase in body temperature due to the elevated temperature of the surroundings. The present invention will be described with particular reference to garments for use by athletes to minimise heat stress and possibly enhance athletic performance. However, the applicability of the present invention is not restricted to such use, and a broad range of other practical applications are envisaged.

Heat stress is the failure of the cooling mechanisms of the body to dissipate sufficient heat energy to normalise the body core temperature (about 37° C.). Heat stress can lead to a reduction in reaction time, reduced energy/lethargy, attention deficit and muscle memory loss. This can lead to decreased efficiency, loss of functionality, decreased personal comfort and, at worst, reduced personal safety.

To optimise body function it is therefore important to maintain body temperature within safe levels during physical exertion, especially in high temperature environments. This is particularly important in sports where the athlete is likely to undergo some form of pre-event/match warm-up routine and/or be required to remain in a high temperature environment for a prolonged period, for example between events in track and field athletics. Indeed, research has shown that pre-cooling of the body before physical exertion can reduce athletic physiological strain in warm environments, thereby typically improving performance and/or minimising heat stress.

Numerous efforts have been made to adapt clothing in order to provide the wearer with a cooling effect. Much attention has focussed on phase change materials (PCMs) which function by changing physical state in response to temperature changes in the surrounding environment. When the external temperature rises above the melting point of a solid PCM, the PCM melts by absorbing from the surroundings the necessary latent heat. On the other hand, when the ambient temperature falls below the melting temperature of a liquid PCM, solidification occurs with release of stored latent heat.

The ice to water phase change has been relied upon extensively to effect cooling during the melting process. However, in this case the body must also be adequately insulated from the ice in order to avoid discomfort and/or chilling. The need for insulation can add to the overall bulk and cost of a garment relying on this system. Ice is also inflexible and this can lead to the garments being cumbersome and uncomfortable to wear. Cooling performance may also be diminished where the inflexibility of ice impedes intimate thermal contact with the contours of the body.

Inorganic salt hydrates are also commonly used. These tend to be cheap and exhibit favourable heat storage characteristics. However, the salts tend to segregate resulting in a reduction in active volume. Salt hydrates can also exhibit supercooling (delayed on-set of solidification) and tend to be corrosive to metals that are sometimes used in thermal storage systems.

Use has also been made of paraffins waxes, and the like. They tend to be chemically stable, exhibit little or no supercooling effect, are relatively safe and non-reactive. Their flammability may be reduced by suitable containment. However, conventional commercially available waxes tend to exhibit low thermal conductivity in the solid state and a broad temperature range over which the complete phase change is observed. The result is either very slow or incomplete phase change and poor sensitivity. High volume changes can also accompany the phase change and this can cause containment problems.

It is also known to microencapsulate PCMs into fibres, fabrics, foams and/or coated surfaces to impart thermoregulatory properties to textiles. However, the microcapsules tend to be small with the result that the thermal capacity of the PCM is relatively low. Water, such as perspiration, may become trapped within the bulk of the textile and this can also be to the detriment of the thermal capacity of the PCM.

Embodiments of the present invention seeks to address the problems described above. The invention takes the form of a number of different embodiments. These embodiments may be employed independently or in any combination.

In one embodiment the invention resides in the use of a PCM which has been specifically formulated in order to have suitable thermoregulatory characteristics. According to this embodiment the invention provides an article of clothing comprising a PCM which is a blend of at least two compounds and which has a melting point of from 5 to 30° C., for example from 10 to 30° C., and a melting temperature range of from 1 to 5° C.

Advantageously, the PCM used has a melting point of from 5 to 30° C. In the context of the present disclosure the term melting point is intended to denote the temperature at which the PCM begins to change phase. It will be appreciated that for a given solid material complete melting from a solid to a liquid does not take place at a single discrete temperature but over a temperature range. In accordance with the embodiment described this temperature range is from 1 to 5° C.

The fact that the PCM has a melting point of from 5 to 30° C. means that it can be provided in close thermal relation with skin without the need for additional insulation to ensure comfort. This leads to an increase in overall thermal exchange efficiency and sensitivity. This also means that the article of clothing can be less bulky due to the absence of need for insulation between the PCM and the wearer's skin. The article of clothing in accordance with the invention is usually worn in direct contact with the skin or in contact with a layer of clothing covering the skin. In the latter case the clothing is preferably thin and close fitting so as to offer minimum resistance to heat transfer between the skin and the PCM as used in accordance with the present invention.

The lower limit of the melting point range for the PCM is selected because at lower melting points the article of clothing may feel uncomfortably cool, especially when the PCM is provided in close thermal relationship with the wearer's skin, as envisaged. The upper limit of the range is selected because there needs to be a sufficiently large difference between the skin temperature of the wearer and the melting temperature of the PCM for efficient cooling. In general, the skin will be cooled provided that there is a temperature gradient favouring the flow of heat from it to the PCM. The temperature difference between the skin and the PCM should be sufficiently large to ensure rapid heat transfer. With a larger temperature gradient, less blood has to flow to the skin to achieve a given degree of cooling. However, this tends to cause chilling. If the temperature of the PCM is too low, skin blood flow may be reduced to such an extent as to make transfer of heat from the body core to the skin inefficient and the body will attempt to retain heat. This would be counterproductive. In practice PCM selection and/or article design is likely to vary from individual to individual taking such considerations into account. The extent and duration of cooling on the performance of a particular individual is also obviously a primary concern.

Preferably, the melting point of the PCM is from 5° C. to 24° C. The preferred melting temperature and melting temperature range are intended to optimise the desirable characteristics of the PCM.

The PCM chosen will have a melting temperature (operating temperature) below skin temperature at the ambient temperature conditions at which the cooling garment of the invention is to be worn. As a result the PCM is thereby able to decrease skin temperature to below the normal 32° C. The result is a small but suitably significant decrease in core body temperature. The lowering of the core body temperature through heat uptake by the PCM is intended to reduce the occurrence of heat stress and, possibly, lead to an enhancement in athletic performance.

The PCM used in this embodiment has a melting temperature range of from 1 to 5° C. Desirably, melting of the PCM takes place over a very narrow temperature range as this ensures rapid heat absorption during PCM melting. The result is a rapid thermoregulatory response. Once the phase change to liquid has been completed effected, this also means that the PCM may be re-solidified rapidly, ready to be used again. This would be especially useful in situations where the article of clothing is worn for only a brief period and/or where the cooling ability of the article of clothing must be regenerated quickly by cooling. Preferably, the PCM has a melting point range of from 1 to 4° C., for example from 1 to 3° C.

In practice the cooling potential of a given PCM may be gauged by reference to its heat of fusion, typically measured as being the amount of energy required to melt unit mass of the PCM. The heat of fusion for a PCM may be determined by conventional techniques. PCMs useful in practice of the present invention generally have a heat of fusion of 150 kJ/kg to 250 kJ/kg. It will be appreciated that a PCM that has a higher heat of fusion has the capacity to absorb more heat per unit mass. This means that to achieve the same maximum heat load (heat of fusion×mass) less PCM of high heat of fusion will be required when compared with a PCM having a lower heat of fusion. The heat of fusion of the PCM is however only one factor that is likely to be considered in PCM selection. The melting characteristics of the PCM as described above are obviously also an important consideration.

PCMs exhibiting suitable melting characteristics may be formulated by blending (at least two) compounds to provide the desired combination of properties and a significant aspect of the present invention is the tailoring of the PCM properties for the intended application, depending upon amongst other things, the extent of cooling required, for instance based on the local temperature environment, the period for which the article of clothing is likely to be worn and/or the period over which the article of clothing is likely to be available for re-activation/regeneration of the cooling functionality.

The PCM usually comprises a mixture of alkanes (paraffins) typically having from 5 to 20 carbon atoms. The alkanes are usually predominantly (at least 95%) straight chain. Certain commercially available mixtures of such compounds will not include suitable proportions of constituents to achieve the PCM characteristics described. It may therefore be necessary to isolate particular fractions of the mixture in order to produce a PCM having suitable properties.

Typically, the mixture of n-alkanes is made up of compounds having a relatively low range of carbon number distribution. This is likely to result in a PCM having a suitably low melting point range. For example, the PCM may comprise a mixture of predominantly (at least 90%) C10-C20, or C15-C20, n-alkanes. Preferably, the mixture comprises predominantly (at least 90%) C14-C18 or C16-C18, n-alkanes. Fractions of alkanes (narrow cuts) may be isolated by selective removal techniques such as fractionation and by the use of selective adsorption, for instance using a molecular sieve. Alternatively, useful PCMs may be formulated by blending high purity n-alkanes which are commercially available. The proportions of the components may be adjusted as necessary to tailor the properties of the PCM. The intention in accordance with the invention is to use relatively small quantities of the PCM due to the enhanced efficiency of the PCM and the contribution of various other embodiments described herein.

Suitable PCMs comprising a mixture of n-alkanes and having the desired array of characteristics are also commercially available as such, for example, from Rubitherm under the designations RT 2 and RT 20, and from Astorstat Thermostat. Waxes and high purity single n-alkane products (for formulation of the PCM by blending) are available from Haltermann Products, Alfa Aesar, Apratim International and Sigma Aldrich

The use of Rubitherm RT 2 and RT 20 have been found to be well suited to practice of the present invention. The RT 2 product is predominantly tetradecane and has a melting point of about 6° C. and exhibits a melting point range of +2° C. It also has a reported heat of fusion of 214 kJ/kg. Given the melting point of this PCM, a freezer is usually required in order to “activate” it prior to use. The means of activation will depend upon the time available. The RT 20 product consists essentially of heptadecane and octadecane with small amounts of tetradecane, pentadecane, dodecane, nonadecane and eicosane. The product has a melting point of about 22° C., a melting point range of ±2° C. and a reported heat of fusion of about 172 kJ/kg. Depending upon the length of time available, the RT 20 product may be “activated” prior to use by storage in an air-conditioned room (below about 18° C.), in a refrigerator or in a freezer.

Preferred characteristics of the PCM include:
1. high heat storage to ensure the minimum possible amount of PCM is required to absorb the wearer's thermal load;
2. heat storage and release takes place at relatively constant temperature to ensure quick responsiveness to the wearer's skin temperature and a chilled atmosphere (on regeneration of cooling functionality);
3. low volume change during phase change so that suitable containment is not excessive resulting in surface area that cannot be used for heat transfer (e.g. less than 17% expansion on complete melting)
4. high crystallinity to ensure good kinetic properties in the reversibility of the PCMs transition;
5. no significant supercooling—on PCM regeneration, it is necessary that all absorbed energy is released to the cooled atmosphere. When supercooling occurs, crystalline structures in a thermodynamically metastable state are formed, complete solidification of the melt occurs slowly, if at all, resulting in extended regeneration times and losses in the amount of stored heat energy;
6. ecologically safe and non-toxic; and
7. recyclable.

The PCM is usually provided in the article of clothing in the form of discrete packs or pouches. Conventional packaging systems and arrangements of the pouches in the article of clothing may be employed. However, another embodiment of the present invention relates to the way in which the PCM is encapsulated for use in the article of clothing.

As noted above, containment of PCMs can be problematic. This is because the material used for packaging the PCM must exhibit a number of beneficial properties. The material used must be sufficiently strong so that it is puncture, tear and rip resistant. Desirably the material is also flexible to maximise surface area contact with the surface to be cooled. The strength and flexibility of the material are also important properties in avoiding leakage due to volume expansion when the PCM changes state.

It is also desirable that the packaging material for the PCM is essentially inert to the PCM contained and is neither corroded nor its properties significantly affected. Some PCMs are able to permeate certain layers, resulting in sweating of the PCM over prolonged use. Equally, the material should prevent ingress of external species, such as water vapour (in the form of sweat), into the body of the container and thus into the PCM. The material should also preferably be gas (e.g. O₂) impermeable as gas ingress can also adversely affect the PCM properties. It is also desirable that the material used for packaging the PCM may be suitably sealed to prevent PCM loss and aid manufacture. Preferably, the material used will be heat sealable.

The material must also exhibit suitable thermal transfer properties to maximise heat transfer across it and from the PCM. This will aid efficient cooling of a wearer in use and rapid re-generation of cooling ability when the PCM is chilled.

In accordance with the present invention it has been found that these desirable properties may be achieved using a laminate rather than a single layer of material. Thus, the present invention also provides a PCM encapsulated by a laminate film, wherein the laminate film comprises an outer heat-sealing layer and an inner layer which is impermeable to the PCM. Herein the terms “inner” and “outer” denote the relative position of the layers of the laminate relative to each other and the PCM. In the embodiment described the outer layer is remote to the PCM relative to the inner layer. Additional layers may be present however and the terms “inner” and “outer” are not intended to define the position of the layers in absolute terms.

In a preferred embodiment the laminate is a three-layer film comprising a layer which is impermeable to the PCM interposed between heat sealing layers. This arrangement offers more flexibility in manufacture of the encapsulated PCM because both outer layers of the laminate are heat-sealable.

In the embodiments described the materials chosen for each layer may individually or in combination provide desirable properties such as strength, puncture, tear and rip resistance and flexibility. The use of a laminate means that these properties need not be provided by any one material. Obviously, the laminate itself should exhibit the desired level of flexibility and thermal conductivity and the material for each layer, and the thickness of each layer, will be selected accordingly. Desirably the overall thickness of the laminate is from 30 to 150 μm, more preferably from 50 to 100 μm.

A particularly preferred laminate for use with the kind of PCM defined above comprises a layer of (biaxially oriented) nylon interposed between layers of LLDPE. The nylon provides structural rigidity and imparts good chemical and abrasion resistance. The LLDPE offers water barrier properties and is heat-sealable. The nylon layer is typically from 10 to 50 μm thick and the LLDPE layers from 50 to 100 μm thick. Preferably, the nylon layer is about 15 μm thick and each layer of LLDPE about 51 μm thick. Such laminates are commercially available, for example from Cryovac under the designation RA463.

The encapsulated PCM is typically prepared by forming a pouch/pack of the laminate which is sealed around the edges and provided with a suitably sized unsealed section to allow the PCM to be inserted into the interior of the pouch. Usually the edges are heat sealed by conventional techniques. Generally, the greater the seal width, the stronger the seal. One skilled in the art would have no difficulty in determining a suitable seal width to use given the intended field of application for the encapsulated PCM. Preferably, the seal width is minimised for economy of material used.

A pre-moulded block of the PCM of pre-determined volume (based on the volume capacity of the pouch) may be inserted into the pouch and sealed in place by heat sealing. In this case the prevailing temperature conditions are obviously such that the PCM is in solid form. As an alternative the prevailing temperature conditions at which the pouch is filled may mean that the PCM is a gel or liquid. In this case the gel or liquid is delivered into the interior of the pouch prior to heat sealing. Typically, the PCM is frozen prior to heat scaling. This can be beneficial in avoiding unwanted flow of the PCM that would cause final seal contamination during the heat sealing aspect of the process.

Air trapped within the pouch can cause insulation interference in the heat transfer process and sealing of the pouch is therefore preferably undertaken in a vacuum. The volume of PCM included in the pouch is calculated taking into account the volume capacity of the pouch when sealed and the volume expansion that the PCM will undergo on phase change. This is done to avoid rupturing of the pouch. A variety of configurations of encapsulated PCM may be prepared in this way. As will be explained below with particular reference to the figures, a number of pouches of different size are usually employed in a cooling garment in accordance with the present invention. The dimensions and configuration of the pouch will determine the surface area of the PCM available for heat transfer. Preferably, this surface area should be as large as possible taking into account size constraints based on the intended location of the pouch in the cooling garment. Thus, it may be possible to use relatively large pouches on planar areas of the garment, such as front or back pieces, whereas smaller pouches would be required in less planar areas, such as arm or collar sections. In practice selection of pouch dimensions for a particular region of the garment will also be predicated by the flexibility required of that region. This is discussed in more detail below.

Generally, the pouch takes a regular geometric shape (usually a rectangle or square). When filled with PCM the pouch is slim and usually has a maximum thickness of less than 2 cm, preferably less than 1.5 cm, more preferably less than 1 cm. Typically, the pouch is from 50 to 150 mm long and from 25 to 50 mm wide. Usually, pouches having different sizes will be used in a single cooling garment. The width of the (heat) seal is the minimum necessary to achieve the required seal strength for a given application. By way of example, the pouches may have approximate dimensions 110 mm×50 mm (and having an internal volume capacity of about 50 ml) and 75 mm×50 mm (and having an internal volume capacity of about 30 ml). With such pouch dimensions the width of each seal is typically 5-7 mm. These widths are included in the dimensions given.

The volume of the PCM used may vary depending upon the heat of fusion of the PCM. To achieve the same cooling performance (in terms of maximum heat load of the PCM) it is possible to replace a particular volume of one PCM with a reduced volume of another PCM having a higher heat of fusion. However, the pouch dimensions in terms of the surface areas over which thermal transfer takes place may actually remain the same, especially where the pouches are designed or configured to cover particular areas of the body. A suitably sized or configured surface area for thermal transfer will also be required, irrespective of the heat of fusion of the PCM used.

The amount and type of the PCM to be used in a given situation may be determined (in general terms) based on the total heat loss that must be accommodated over the period for which the cooling garment is to be worn. For example, if it is known that the total heat loss associated with a particular activity is 500 kJ it can be estimated that to accommodate this will require the use of 2 kg of a PCM having a heat of fusion of 250 kJ/kg. The same amount of heat loss may be accommodated with a PCM of lower heat of fusion but then a larger amount of PCM will then be required. Thus, if a PCM having a heat of fusion of 200 kJ/kg is used, the amount required will be 2.5 kg. In reality the amount of PCM required will be slightly higher than the theoretical amount that is calculated since the process of heat transfer to the PCM is not ideal, and the heat loss from various parts of the body is likely to vary. This can be accounted for in design of the cooling garment. In this way it is possible to tailor the cooling garment to an individual's needs or to a range of needs based on the required heat loss to be accommodated.

The PCM should be “activated” prior to use and by this is meant the PCM must be in a form that is suitable for absorbing thermal energy. The PCM may be activated by cooling and the rate of activation will depend upon the PCM used and the cooling to which it is subjected. For instance, a cooling garment including the RT 2 PCM may be activated by storage in a freezer (e.g. about −18° C.). In contrast the RT 20 may be activated using an air-conditioned cool room (about 16° C.), a fridge (about 4° C.) or a freezer (−18° C.). The amount of time required for complete activation will depend upon the number of PCM pouches in the garment and desirably the garment should be arranged so that the PCM pouches enjoy the maximum cooling effect. It may be appropriate to expose as much of the inner pocket layers/areas to a cold chamber for maximum exposure of the thermal exchange surfaces of the PCM pouches. It is of course possible to remove the PCM pouches from the garment for activation but removal and reinsertion of the pouches can be very time consuming.

Another embodiment of the invention relates to the form in which the PCM is incorporated into a cooling garment. Conventional thinking suggests large volumes of PCM material should be included in large, continuous components. However, this can lead to the resulting garment being bulky and inflexible. Efforts to alleviate these problems have focussed on dividing large unitary components into segments/compartments which are joined by integral, flexible webbing. Efficiency of cooling is directly dependant on heat transfer from the body by conduction via skin contact and the use of numerous relatively small segments/compartments is intended to facilitate this. However, a further problem with the use of even this type of construction is that the materials used to contain the PCM (and which also form the webbing) are not water permeable and perspiration can accumulate on the body between the skin and the PCM-containing structure. This can lead to discomfort and detract from the overall efficiency of the cooling garment.

It has also been suggested to provide the PCM in pouches for insertion into suitably sized pockets provided in the garment. However, these pouches have also tended to be bulky and are not especially efficient. An embodiment of the present invention is based on the appreciation that a cooling garment may be prepared in which the PCM is contained in a number of individual and slender pouches which enable a flexible garment with good cooling efficiency to be prepared. The use of such pouches means that there will be numerous spaces between adjacent pouches and this can allow production of a breathable garment to be prepared, subject of course to selection of suitable material to occupy the spaces between the pouches.

In this embodiment the individual pouches are typically characterised as having a heat exchange surface area to volume ratio of from 1.06 to 1.20, for example from 1.06 to 1.10. For the entire garment (including any and all head/neck/chest/shoulder/back/sleeve portions), the heat exchange surface area to volume ratio is about 1.78. In this context the volume referred to is actually the volume of PCM material included in the pouch. The surface area proximal the surface to be cooled (skin) should be as high as possible whilst bearing in mind that the resulting garment should retain high flexibility. Preferably the pouches take the form of elongate rectangular members, the dimensions of which may be varied depending upon the intended location of member within the garment. It is also possible to manipulate the PCM loading and dimensions of the pouch in order to provide increased cooling efficiency over localised areas of the body where increase heat generation occurs, such as the head, chest, shoulder, neck and back. It is preferred to provide enhanced cooling to the shoulders and back due to the high heat loading observed at these locations during exercise.

If the PCM pouches are sized appropriately, it is possible to arrange the pouches over a large surface area of a garment whilst retaining adequate flexibility. The nature of the PCM encapsulation and the presence of spaces between adjacent pouches contribute to this. The pouches should be shaped in order to maintain optimal contact between the heat exchange surface of the pouch and the wearer's skin. The pouches may also be shaped and positioned within the garment so as to be in intimate contact and aligned with major blood vessels in the wearer's body. This can also enhance cooling performance.

The PCM pouches may be inserted into pockets within the garment and sealed therein, either permanently for example by stitching or removably for example by fasteners such as zips and velcro. The material from which the garment is made is preferably lightweight and breathable, and shaped so that in use the PCM pouches will be in close proximity to the wearer's skin. The arrangement of PCM pouches should preferably afford the wearer ease of movement and offer a high level of comfort.

The PCM pouches are used in sufficient numbers to extend over a large area of the garment. It is desirable not to include any pouches at areas required to be flexible, such as elbow portions in a jacket, although suitably sized pouches may be arranged around such areas. It is also desirable to leave some gap between adjacent pouches to allow the garment to be breathable (as noted the textile used for the garment is preferably breathable). The pouches do exhibit a degree of flexibility and can be deformed (in use) to provide increased comfort. Fitting of the pouches to body contours in this way can also improve the efficiency of thermal transfer. The arrangement of pouches in an article of clothing is preferably designed by reference to infrared thermal imaging of the body of the intended wearer during pre-event, intra-event and/or post-event (cool down) periods, depending upon the intended use of the clothing.

The garment may take the form of a jacket, trousers, shorts, hood, hat, gloves etc, depending upon the intended field of use. It will be appreciated that the garment should be designed so as to allow the wearer comfort and ease of movement. Thus, where cooling of the torso is required but the wearer's arms must be free to move (such as in rowing or shooting), the garment takes the form of a sleeveless jacket (referred to herein as a vest). Alternatively, it may be possible to use a jacket with sleeves that may be removed, as might be required. Typically, when the garment is a jacket (with or without long sleeves), the jacket also preferably has a collar portion and/or removable hood. The sleeve portions of the jacket may include a zippered portion (or the like) running along a seam of the sleeve from the wrist to the elbow, and possibly beyond. This is intended to allow the jacket to be put on and/or taken off with ease. The same arrangement maybe used in trousers, the zippered portion (or similar fastener) being provided along a seam running from the bottom of the legs to the knee, or beyond. This also allows the trousers to be put on and/or taken off over footwear. When a hood (or hat) is worn it may be important for the wearer to be able to hear easily, for example to listen to training instructions. In this case the hood (or hat) may be suitably shaped around the ears or include holes over the ear regions.

In one embodiment the cooling garment may be a full body suit with removable sleeve and/or leg and/or hood portions. Such portions may be removed as required to meet an individual's particular cooling and/or comfort needs.

PCM pouches are usually fitted into all portions of the garment with particular attention on areas which in use are likely to come into proximity with body sites of high heat dissipation. Typically, the PCM material is concentrated in the garment to provide a high level of cooling to the chest, back and/or shoulder areas. The size of the pouch and thus the amount of PCM material contained may be varied accordingly. To aid flexibility the PCM pouches may be provided in a rib-like arrangement across the front and back of the garment (jacket in this case). The pouches should not extend over flex points, as noted. Regarding jacket design, the garment is compact and strategically laid out pouches allow a higher concentration of PCM loading than conventional ice jacket without compromising the mobility and comfort of the wearer.

The cooling garment in accordance with the present invention may comprise inner and outer shells. The inner shell is adapted to receive the PCM pouches in a suitable configuration to optimise cooling efficiency. The outer shell overlies the inner shell (and is usually attached thereto) and is intended to improve the overall aesthetic appearance of the garment as well as providing desirable functional properties such as wind and rain resistance. The inner shell may be formed of cotton wadding and the outer shell of a blend of natural and synthetic fibres such as nylon and cotton (available commercially as Coolmax Aquator, 51% nylon, 49% cotton). Desirably, both shells are lightweight. The outer shell may include surface decoration, motifs, advertisements, and the like.

To maximise heat transfer it is preferred that the PCM-containing pouches are suitably arranged in a garment. It is also preferred that the pouches are in intimate contact with the wearer's body with minimum insulation between the body and the PCM. In one embodiment of the invention the garment comprises an elastic material and is close-fitting when worn. In this case the elasticity of the garment material assists in maintaining the PCM and wearer's body in intimate contact, thereby potentiating thermal transfer.

Additionally, or alternatively, the same effect may be achieved by suitably positioning fittings that allow the thermal exchange surface of the garment to be brought into close contact with the wearer's body, or parts thereof. Air present between the wearer's body and the thermal exchange surface can act as an insulator thereby impeding the desired transfer of thermal energy. The fittings referred to are intended to reduce or avoid this effect. In the case of a jacket, these fittings may take the form of adjustable belts arranged around the torso portion of the jacket. When worn these belts may be adjusted to ensure a suitable fit to maximise transfer of heat from the body to the PCM. If the jacket includes arm portions, these may include adjustable belts or elasticated bands around the arm portions, to ensure that the same effect may be achieved. Similarly, to ensure a hood/hat is a suitably close fit for optimum thermal exchange, the hood/hat may include ribbing, elasticised elements and/or a draw-string, or the like.

The garment should also be designed to take into account the wearer's posture, and likely movement, during intended activity. For instance, in sports such as rowing where the athlete is constantly in a sitting position, with knees moving up to and away from the chest, the garment should be adapted to avoid bunching or bulging thereof. This may be achieved by designing a garment that does not extend below the waist and that fits the torso closely. In this embodiment garment design is also likely to vary from individual to individual.

Preferably, the garment is also designed to be put on and removed rapidly. In this way the efficiency of the garment is maximised and the cooling effect brought about by its use is diminished to the least amount possible. To this end the garment may include zippers, velcro fastenings, press studs, and the like.

In one embodiment of the invention the garment includes pouches containing different types of PCM depending upon the location of the pouch within the garment. In areas of the garment corresponding to regions of the body of high heat loss, the pouch(es) may contain a PCM having a high heat of fusion. In contrast, in areas of the garment corresponding to regions of the body is where heat loss is lower, but nevertheless significant, the pouch(es) may contain a PCM having a lower heat of fusion. In this way different parts of the garment can be tailored in order to match the specific heat loss characteristics of an individual based on the physiology thereof.

In this particular embodiment the cooling garment may include ice packs in addition to pouches containing the PCM as described. However, in this case, care must be taken to ensure that the ice packs are sized, insulated and/or positioned appropriately to avoid any unpleasant thermal sensation/thermal shock when the cooling garment is worn. Here it may be appropriate to position the ice packs (or pouches with PCM of low operating temperature range) along or adjacent the spine region of a cooling jacket or vest. In a preferred embodiment the cooling garment includes pouches containing the RT 2 PCM and pouches containing the RT 20 PCM. In this case the pouches containing the RT 2 PCM tend to be concentrated at areas in the garment corresponding to high heat loss, such as the torso of a jacket or vest, with the RT 20 PCM being used in peripheral areas, such as in sleeve portions.

The present invention is believed to have significant applicability to the thermoregulatory control of athletes/sportspeople. As noted, it is known that cooling of the body before physical exertion can reduce physiological strain in warm environments and, possibly, enhance performance. In this context preferably the cooling garment of the present invention is employed in order to reduce the core body temperature of an individual such that after completing a suitable warm-up or pre-event routine (without the cooling garment) the core body temperature of the individual is approximately the same, or even slightly lower, than that before cooling was initiated. Use of the cooling garment in this way allows a warm-up or pre-event routine to be completed whilst avoiding any significant increase in core body temperature relative to an initial/relaxed core body temperature (i.e. prior to any cooling). Without use of the cooling garment in this way, the warm-up or pre-event routine may result in an increase in core body temperature, and this may compromise subsequent event performance. To achieve suitable temperature regulation according to this embodiment it may be necessary to change garments during cooling prior to the warm-up or pre-event routine if the cooling efficacy of the garment being worn is reduced or exhausted prior to core body temperature being achieved. This should be straightforward, especially if the garment is designed to be put on and removed with ease. Alternatively, or additionally, it may be possible to achieve the desired overall reduction in core body temperature prior to the warm-up or pre-event routine by using the cooling jacket before, after or during application of some other method of cooling the body. For example, an initial reduction in core body temperature may be achieved by use of a cool/cold shower after which a further reduction is achieved using a cooling garment in accordance with the present invention. The same effect may be achieved by exposing an individual to a cool/air-conditioned room with subsequent use of the cooling garment.

In practice, an appropriate regime for garment usage during a warm-up or pre-event routine may be designed based on experimental measurement of core body temperature during the routine, taking into account ambient temperature conditions. It would be preferable in this case for the cooling regimen most effective for a particular individual to be logged under a variety of ambient temperature conditions and/or time periods. In this way the most appropriate cooling regimen may be selected for any given situation based on the prevailing conditions and/or time constraints that are applicable.

The cooling garment may be used to achieve pre-event, intra-event, inter-event and/or post-event cooling of the body.

The invention is not restricted to use in connection with human athletes and may be used to enhance the performance of animals that are raced. Thus, the present invention may also have applicability for use in connection with racehorses and greyhounds. In such cases, the exact design of the garment will vary depending upon the areas of the animal's body where significant heating occurs. This can be mapped by thermal imaging. The garment may take the form of a blanket or throw that is draped over the animal prior to racing and/or during warm-up, or the like. Straps, and the like, may be used to secure the garment to the animal in order to optimise heat transfer.

The invention may also find utility in other areas where deterioration of performance due to elevation of body temperature may have adverse consequences. Thus, the present invention may be used to provide cooling in helmets for civil applications (eg. hard hats, police helmets, fire helmets) and in military applications. In this case the PCM may be integral to the structure of the helmet or be included in some form of headwear worn under the helmet. The invention may also be used to provide a cooling garment to be worn under some form of protective clothing. For instance, a suitably designed cooling garment in accordance with the invention may provide cooling under protective outerwear worn by police, fire and military personnel. Use of the invention in this manner may help to enhance performance or rather, prevent deterioration of performance due to a rise in body temperature.

The invention may also have applicability in medicine where cooling of the body or a part thereof is believed to be beneficial. For example, cooling garments may assist in the management and/or treatment of conditions such as ectodermal dysplasia and multiple sclerosis. Cooling of the head may also be beneficial in the case of head trauma. Suitably designed garments may therefore provide a convenient and useful tool to be used by doctors paramedics and emergency practitioners.

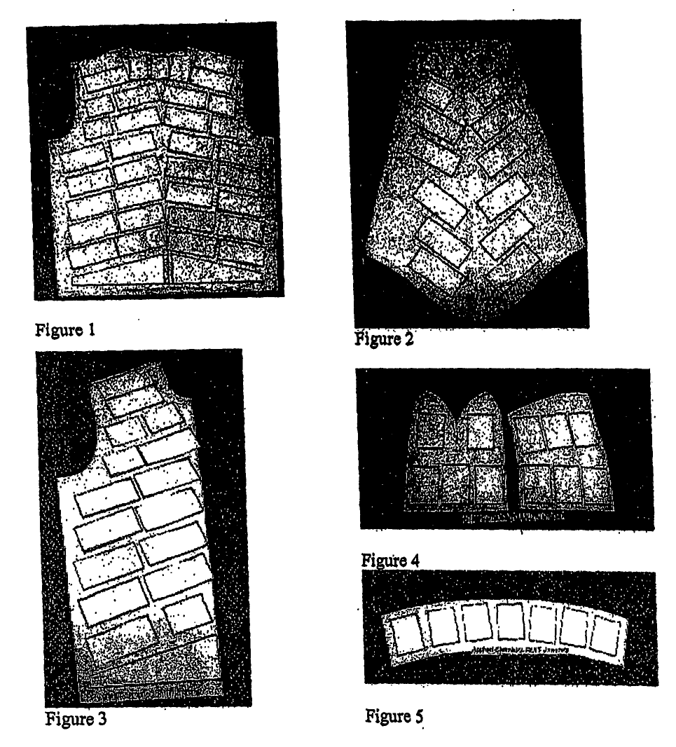

Embodiments of the invention will now be described with reference to the accompanying non-limiting figures. FIGS. 1-11 show schematically how the PCM pouches would be included in component pieces of a cooling garment in accordance with the present invention. The accompanying figures are intended to show the general arrangement of the PCM pouches and should not be taken as being limiting. Variations in pouch size, number and arrangement are of course possible taking into account such things as garment size, desired cooling performance and the desired flexibility and weight of the cooling garment.

FIGS. 1-5 illustrate the arrangement of PCM pouches in component pieces of (the inner shell of) a jacket. FIG. 1 illustrates a back piece having PCM pouches of varying size attached. The PCM pouches are retained in pockets and extend across substantially the entire surface of the piece. The pouches are proved in a rib-like arrangement with small spaces between adjacent pouches to allow the garment to breathe.

FIG. 2 illustrates an arm piece (prior to stitching) and PCM pouches of varying size are positioned to ensure maximum contact with the wearer's skin when the sleeve portion is used in a jacket. Note that the PCM pouches are of smaller size at the “wrist end” of the sleeve (the top part of the piece in the figure). Note also that no PCM pouches are provided at the elbow point, where flexibility is required.

FIG. 3 represents a front half piece. In the embodiment shown two half pieces will be fastenable, for example using a zip fastener. In another embodiment a single front piece may be used and in this case the jacket would be a pull-on type.

FIGS. 4 and 5 illustrate hood and collar pieces respectively.

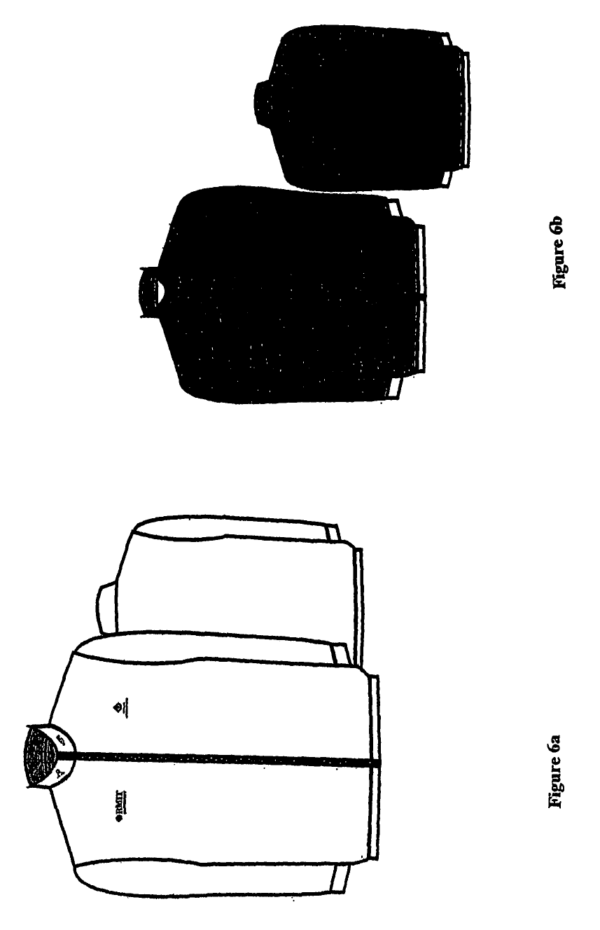

FIGS. 6a and 6b are front and back views of a cooling jacket in accordance with the present invention. FIG. 6a shows the outer shell of the jacket. FIG. 6b shows the inner shell and includes PCM pouches in orientation similar to that shown in FIGS. 1 to 5. The inner and outer shells are essentially the same in shape and overall design, but this is not essential.

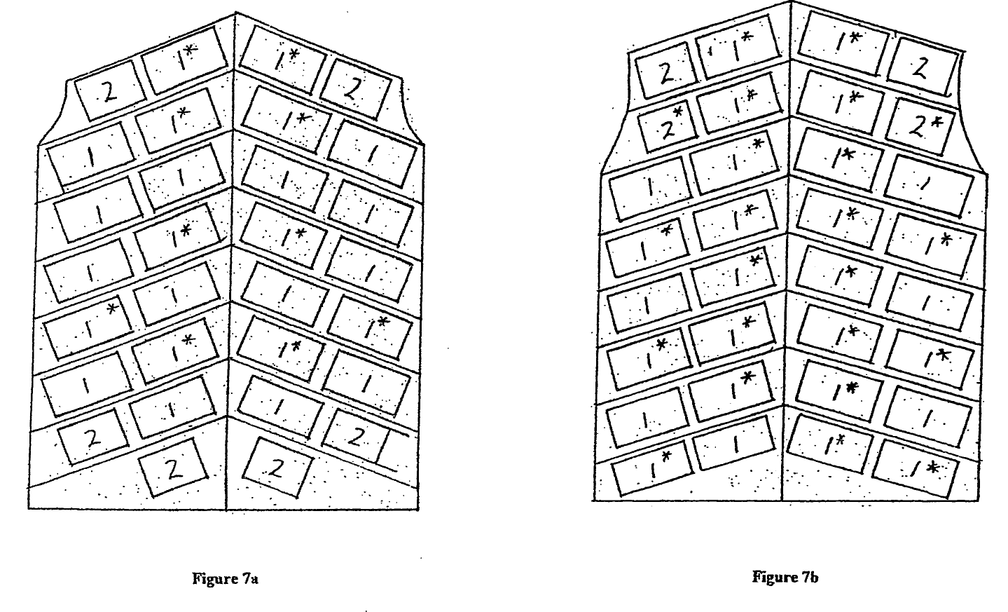

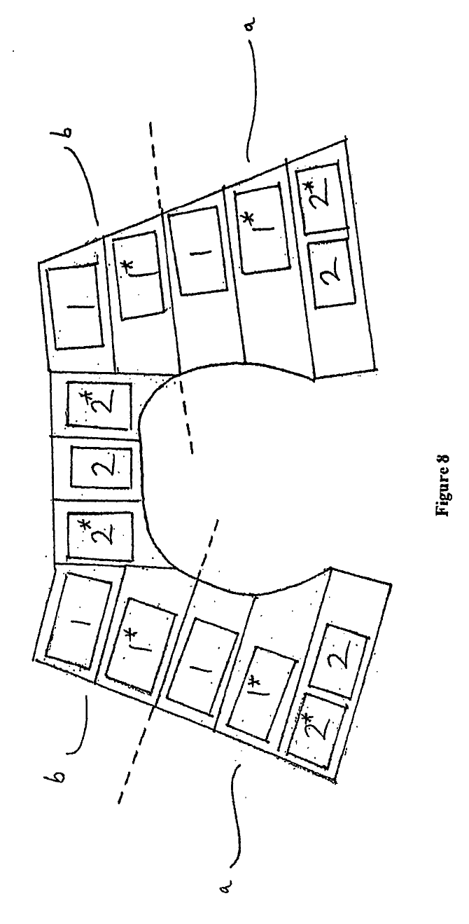

In FIGS. 7-11 the numbers 1 and 2 are used to denote PCM pouches of different sizes. For the size 1 pouch the surface area available for heat transfer (i.e. one side of the pouch) is approximately 5500 mm² with the pouch dimensions being approximately 110 mm×50 mm. For the size 2 pouch the surface area available for heat transfer is approximately 3500 mm² with the pouch dimensions being approximately 70 mm×50 mm. Unless otherwise stated the PCM is the same in each pouch irrespective of size. The pouches are arranged in order to optimise thermal exchange between the wearer's body and the PCM, and to ensure comfort/flexibility in use.

As explained earlier, in an embodiment of the invention the cooling garment may include pouches containing different PCMs and possibly ice packs in addition to the PCM pouches. In this case the PCM with the highest heat of fusion, or the ice, will be located in regions of the garment corresponding to regions of the body where heat loss is likely to be highest. These regions in the cooling garment are identified in the figures by an asterisk (*).

FIG. 7a illustrates a front piece for a (medium sized) jacket or vest. FIG. 7b illustrates the corresponding back piece. Although not depicted the front piece will include a zip fastener, or the like, running down the centre of the piece. In FIG. 7b it will be noted that the asterisks are concentrated in a region corresponding to the spine of a wearer.

FIG. 8 illustrates a yoke for a (medium size) jacket or vest. The broken lines divide the yoke into front sections (a) and a back section (b). In use the yoke will be fixed to the kind of front and back pieces depicted in FIGS. 7a and 7b respectively.

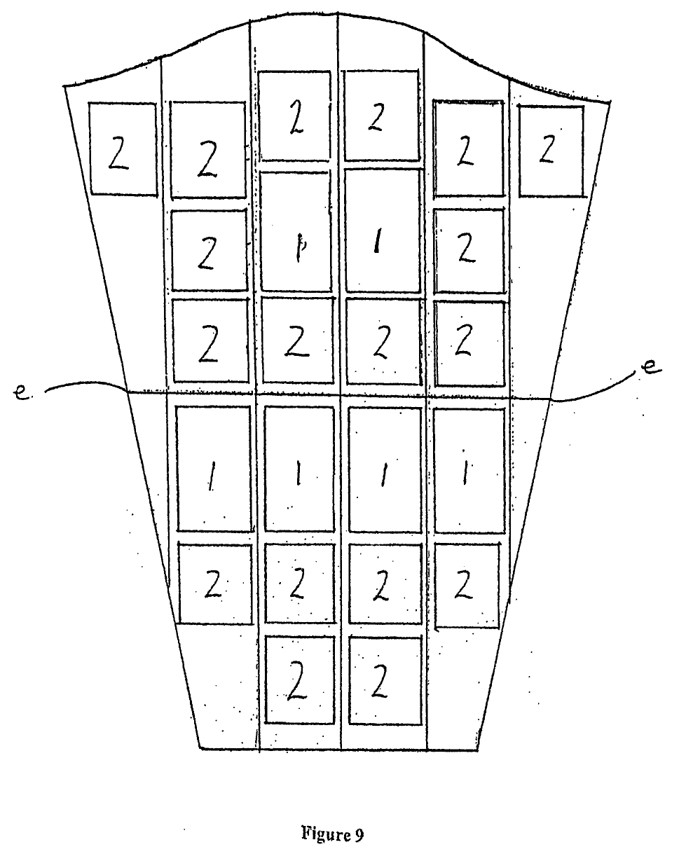

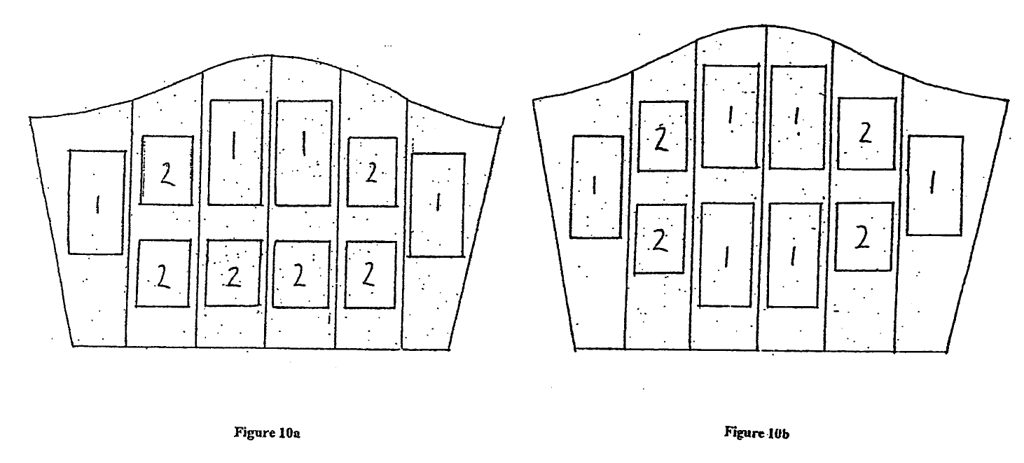

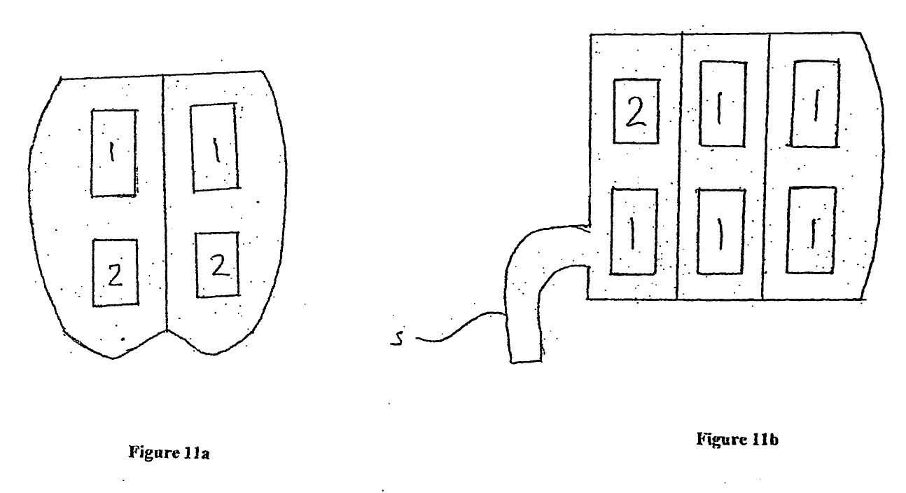

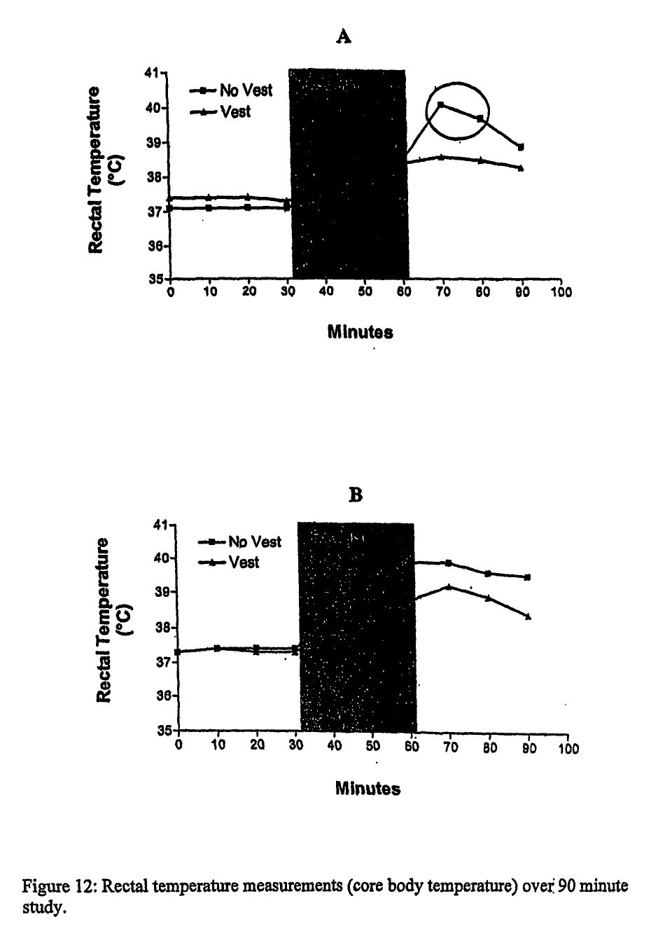

FIG. 9 illustrates an arm piece in the form of a long sleeve. The upper portion of the piece corresponds to the upper arm/shoulder region and the lower end to the wrist region. It will be noted that the PCM pouches are arranged in a fashion such that in use the pouches will run in parallel rows down the arm. It will also be noted that no pouches are provide across a line (e) corresponding to the line of elbow flex in the finished sleeve. This is done to ensure that the pouches do not impair movement. In this respect, the arm piece depicted in FIG. 9 represents a refinement of that shown in FIG. 2. In FIG. 9 the PCM pouches also tend to be aligned with the major blood vessels in the arm, especially in the forearm and wrist regions.

FIGS. 10a and 10b illustrate arm portions for a short sleeve garment. Again, the upper portion of the pieces shown correspond to the upper arm/shoulder region. Short sleeve garments are especially useful where lower arm mobility and flexibility is required to be at a premium and cooling of upper arm area is considered to be significant for the particular application. FIG. 10a shows the kind of arrangement that may be used in a medium to extra large garment, with FIG. 10b showing the kind of arrangement suitable for bigger sizes.

FIGS. 11a and 11b show pieces making up a cooling hood. FIG. 11a represents a top/back piece with FIG. 11b represent a side piece. In practice the hood is constructed from one top/back piece and two side pieces. The side piece also includes a strap (s) appended thereto. Although not shown the side piece may include ear holes to enable the wearer to hear clearly when the hood is worn. When made up the hood may be worn independently. Alternatively, the hood may be fixed to a jacket or vest, possibly removably so.

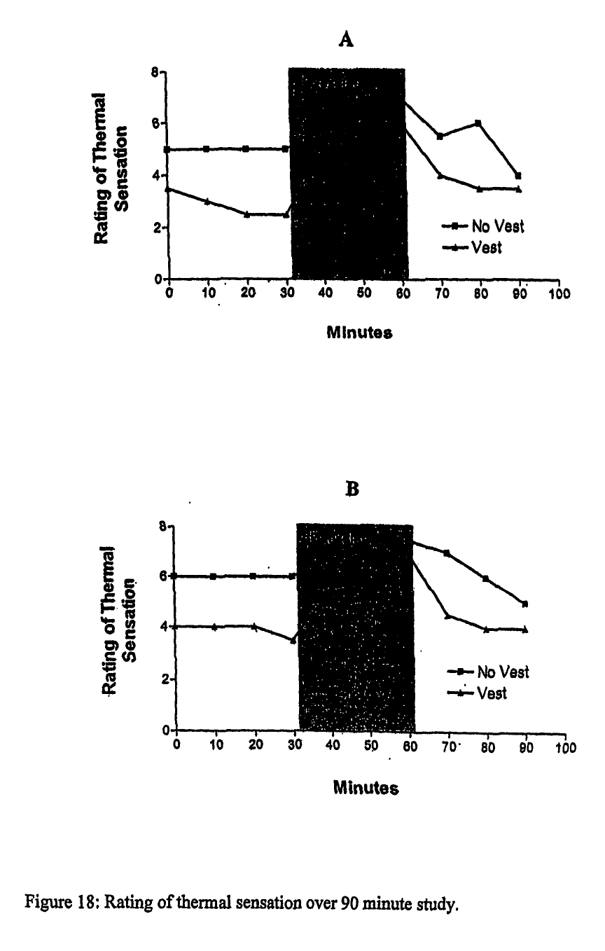

FIGS. 12 to 21 and Figures A to G illustrate experimental results and are discussed in greater detail in the example included below.

The following non-limiting example illustrate embodiments of the present invention.

### EXAMPLE 1

A cooling jacket in accordance with the present invention is prepared as follows. The jacket-like garment is composed of eight panels: the yoke section that covers the top back/front sections, the lower halves of the front and back section (2 left and 2 right sides), 2 long sleeve sections and a high collar. The exterior of the garment is essentially a casing which in this example is Coolmax Aquator (51% nylon, 49% cotton, 240 g in weight) and an interior primarily formed by cotton wadding (85 g in weight).

Within the garment and next to the cotton wadding is located a series of pockets (inner shell) of wool/elastane (96.5%/3.5%, 230 g). This fabric arrangement allows laminate pouches of the PCM to be inserted into this inner shell pockets whilst protection from ambient temperature is provided by the wadding insulation and the Coolmax outer shell. Ribbed cuffs and a bottom band ensure a close fit and protect from ambient warm air prematurely melting the PCM. These flexible heat-dissipating pouches are inserted into the channels from the side of each panel.

The pouches are formed from two heat-sealable transparent laminate sheets. In this example, laminate pouches of about 136 μm thickness and of two sizes (11.6 cm×5 cm and 7.6 cm×5 cm) which were prepared by conventional 3-side heat sealing of two laminate sheets, leaving one side open. Premoulded blocks of PCM (Rubitherm RT 20) of two sizes (7.5 cm×3.5 cm×3.0 cm of volume 52 ml (39 g) and 3.5 cm×3.5 cm×3.0 cm of volume 32 ml (24 g)) were inserted into the laminate pouches and the final seal was made using a vacuum heat sealing unit. Adequate volume was left within the pouch to accommodate volume expansion (about 10% on phase change of the RT 20 product). When filled and remelted to obtain a flat-shaped compartment, the total thickness of the PCM pouch was about 1.5 cm.

Placement of the laminate pouches within the garment is as shown in FIG. 6b and more accurately (with regard to sizes of pouches) in FIGS. 1 to 5. Gaps (1 cm in width) between the channels containing the filled laminate pouches and the small dimensions of the pouches themselves allows all panels to conform to the wearer's body to provide greater comfort, more freedom of movement and breathability when the garment is worn whilst still maintaining a close fit for efficient heat transfer.

The inner shell of fabric (wool elastane) was stitched to form parallel, diagonal channels of a specified width (between 5 and 6 cm). In one example, ten such channels are formed on each front and back panel. FIG. 6b shows thirteen such channels on the front side of the jacket.

The two front panels are secured together via a zipper that extends along the entire length of the garment, from the bottom band up to the top of the collar. A close fitting hood with a PCM arrangement as per FIG. 4, may be attached to the upper collar edge via a zipper, to allow heat transfer from the head. The jacket in accordance with the invention weighed about 4.90 kg without hood and about 5.50 kg with hood.

The cooling jacket was evaluated in preliminary trials at the Australian Institute of Sport (Canberra) using two elite athletes (cyclists). Experiments were conducted with the athletes in an environmental chamber where ambient conditions of relative humidity (60-70%) and temperature (32.5-34° C.) were controlled. One athlete (A) wore a conventional ice vest whilst the other (B) wore the jacket in accordance with the present invention to compare the effectiveness and impact of the new cooling garment on performance.

The ice vest had four large front pockets (2 top located breast-proximate pockets and 2 bottom located pockets) and four back pockets in similar positions to frontside locations. The pouch sizes were 14 cm wide×17 cm long (top pouches) and 14 cm wide×15 cm long (bottom pouches). The pouches were made of a plastic film and filled with water to a volume capacity of 60% followed by freezing. An approximate volume of 2.5 litres of ice was used in the ice vest. The ice vest also included a thin layer of material on the inner surface of the vest to separate the ice packs from the skin surface. The total weight of the ice vest was about 3 kg.

Three to four hours storage time in a freezer is deemed sufficient for regeneration of the ice-vest packs.

In contrast, the cooling jacket of the present invention required 0.5 hours for regeneration after usage (partial melting) in a pre-event application (after use for 0.5 h with an athlete at rest). As for regeneration time in a 4° C. atmosphere, two 11 cm×5 cm laminate pouches (containing 52 ml of Rubitherm RT 20) that were warmed until the contents completely melted were tested. One was placed in a pocket of the textile layers as assembled in the jacket and one was left to regenerate without insertion in a textile pocket. It was found that 90 minutes was sufficient for both pouch contents to solidify into a maleable (flexible) gel ready for re-use.

The two athletes (A and B) were studied over a 90 minute period over which three periods were defined: initial rest time (first 30 minutes, pre-cooling), 30-60 minutes (exercise period, cycle ergometer) and 60-90 minutes (recovery period, post-cooling). The garments were worn only during the pre-event and post-event periods.

Physiological responses such as skin surface temperatures (forearm, thigh, calf, chest), core body (rectal) and heart rate, sweat loss as well as thermal sensation and perceived exertion ratings were collected from each athlete. Temperature data was collected via infrared digital imaging and thermocouples.

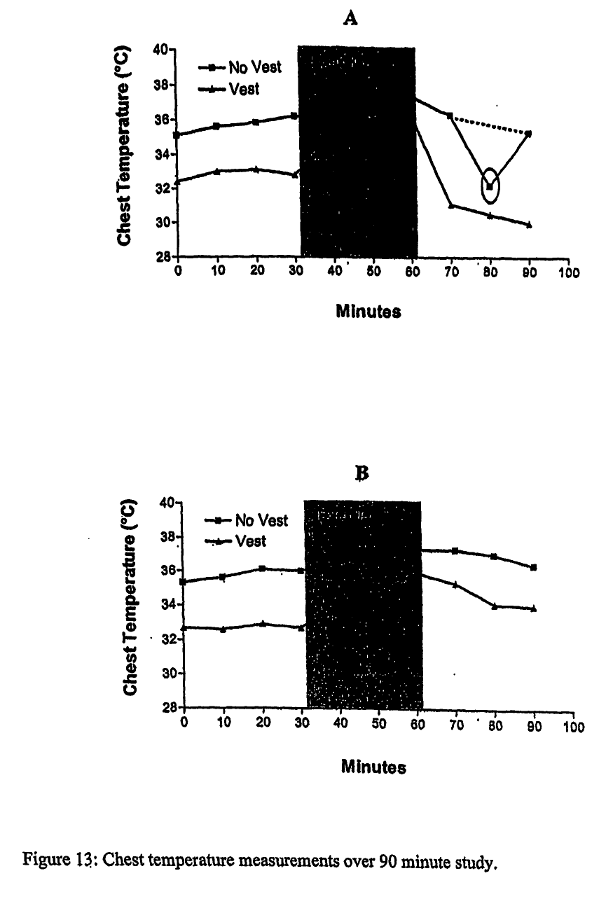

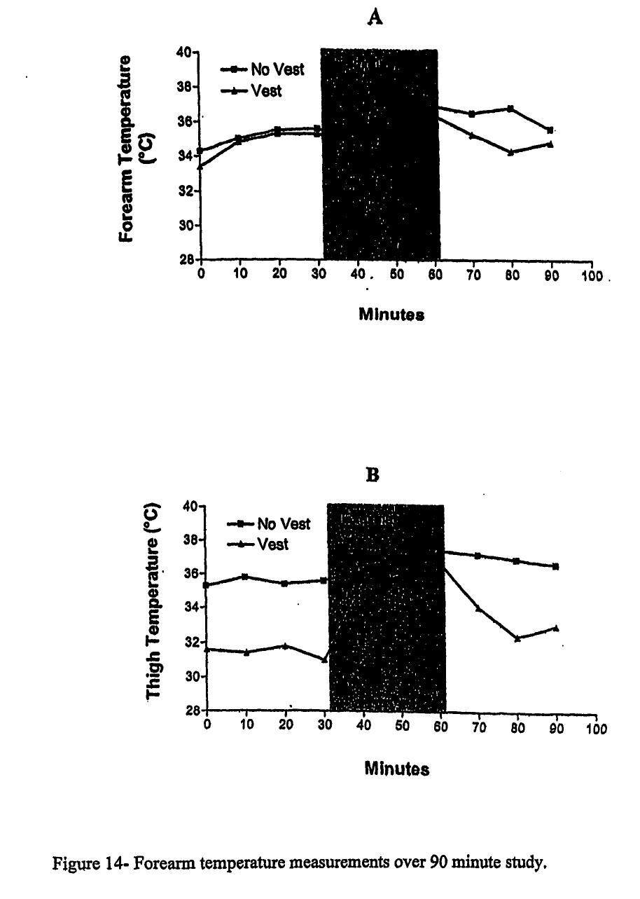

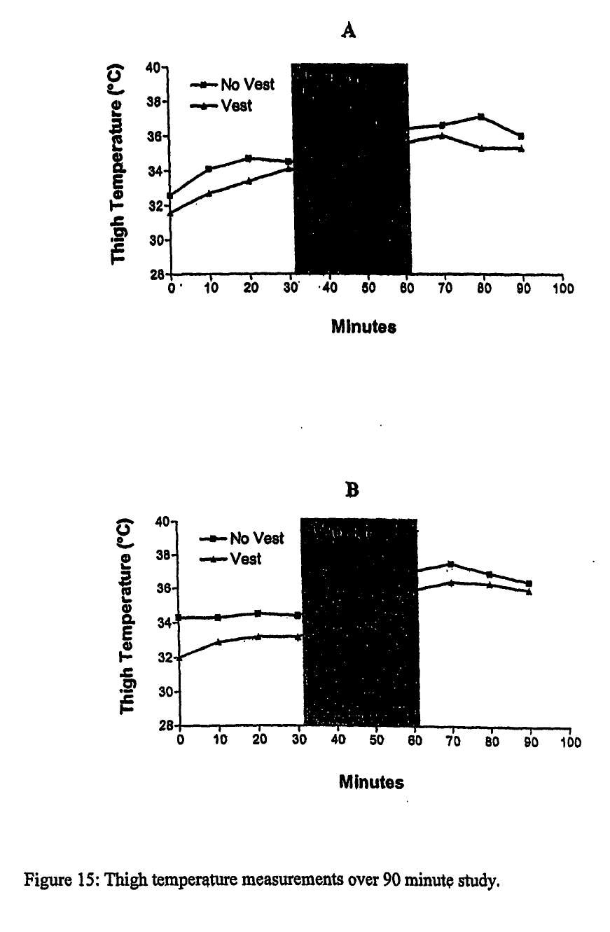

Key improvements and enhancements resulting from wearing the jacket in accordance with the present invention are included in the following discussion. Circled points in the graphs refer to suspected measurement errors in the data collected.
The jacket of the present invention showed a clear reduction in core body temperature (rectal temperature) during the exercise period by 1.1° C. and that this cooling continues into the post exercise period (see FIG. 12).
Both cooling garments appreciably decrease chest temperatures, but this cooling effect extended in to the exercise period with a greater magnitude for the jacket of the invention (keeping temperature 2° C. cooler than with no cooling) than for the ice vest (keeping temperature just 1° C. cooler than with no cooling) (see FIG. 13).
The jacket of the invention reduced the forearm temperature by ˜3.5° C. whereas the ice vest managed to reduce this temperature by ˜1° C. The greatest effect was felt in the first 10 minutes of the exercise period, where it took 20 minutes of exercise for the temperature to reach the non-cooled forearm temperature (see FIG. 14).
Despite both jackets not covering the thighs, the thigh temperatures were significantly reduced for both garments with the new jacket prototype cooling the thighs by 1-2.4° C. during initial pre cooling, 1-2° C. over exercise period and by ˜1° C. during the post-exercise period. Corresponding reductions for the ice vest were lower, ˜1.3° C. cooling over the pre-exercise period, 0.75° C. during the exercise period and by up to ˜2° C. over the post-exercise stage (see FIG. 15).

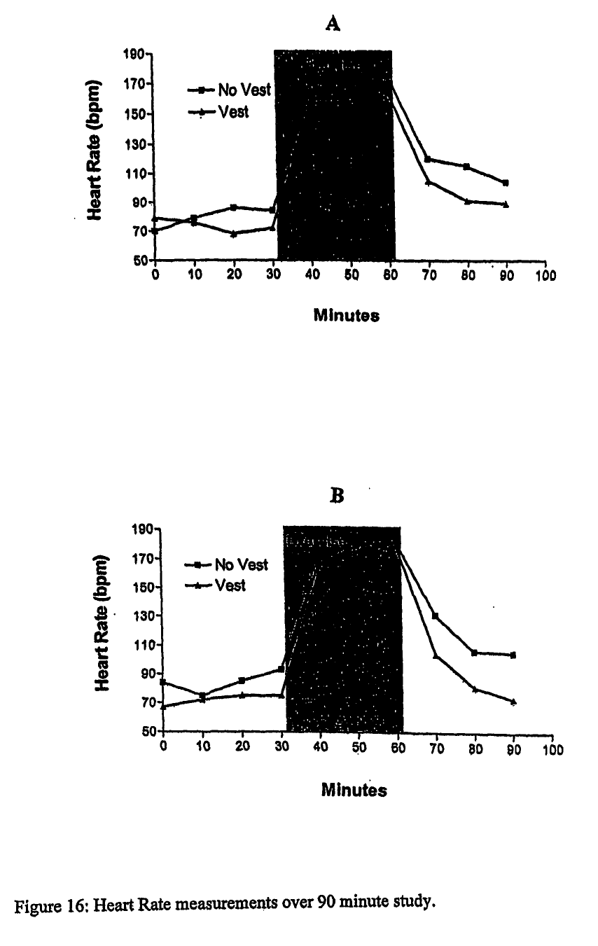

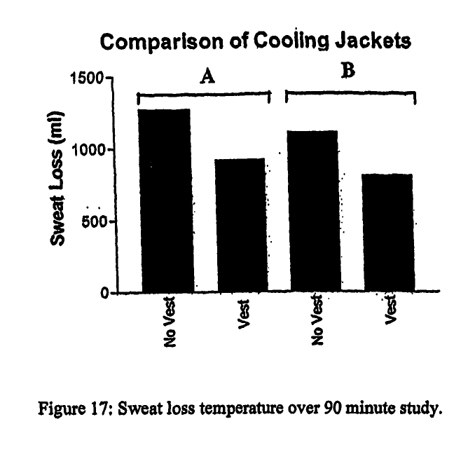

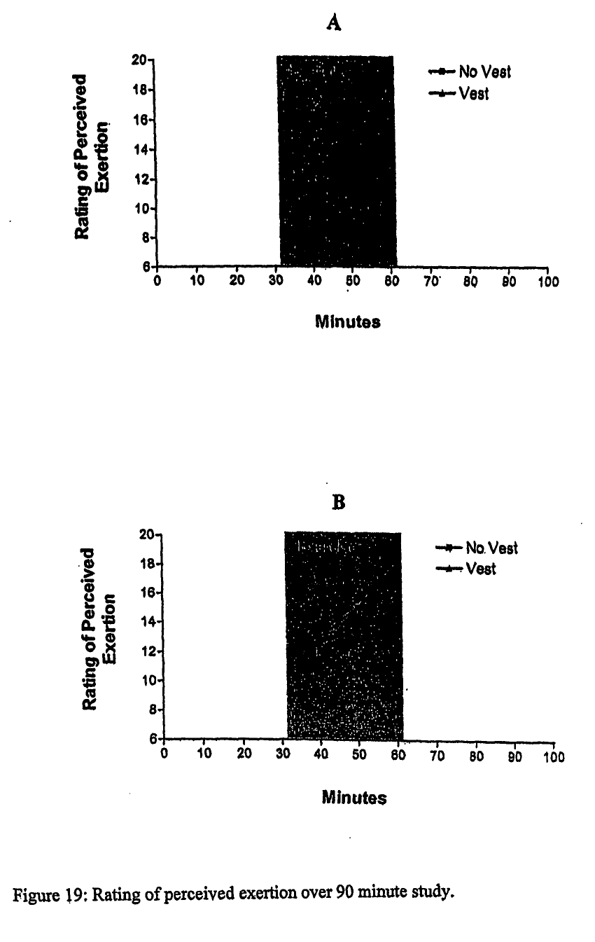

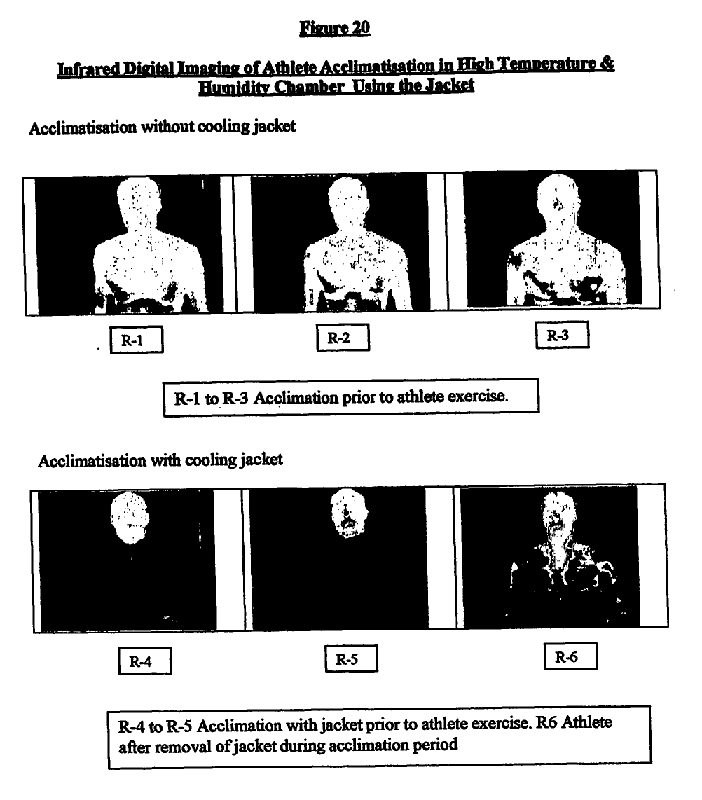

The heart rate was reduced significantly for both cooling garments in pre cooling periods, and moderately in the exercise period. The post-exercise heart rate reduction was greatest for the jacket of the invention prototype in the first 20 minutes of the post-exercise period and to exemplify, within the first 10 minutes of the post-exercise period the jacket of the invention reduced heart rate by 70 bpm while the ice-vest reduced it by 55 bpm. At 90 minutes, the total reduction in heart rate (from post exercise t=0 to t=30 min) was 98 bpm for the jacket of the invention and just 70 bpm for the ice vest (see FIG. 16).
Sweat loss was reduced by ˜28-29% after use of either cooling jacket (see FIG. 17).
Ratings of perceived exertions were lower for both types of pre cooling jacket the reduction in exertion was less for ice-vest cooling than for the jacket of the invention. That is, athlete A reported a slightly larger difference in amounts of exertion with and without pr cooling, i.e, pre cooling with the prototype caused a lowered rating of perceived exertion (see FIG. 19).

The jacket in accordance with the present invention has been shown in this example to reduce the heart rate significantly, have a more enduring cooling effect in the post exercise period, provide significantly lower thigh temperatures despite not covering the legs, provide a lower thermal sensation rating (chilling), and provide a lower exertion rating with respect to no pre-cooling, compared to the conventional ice-cooling jacket.

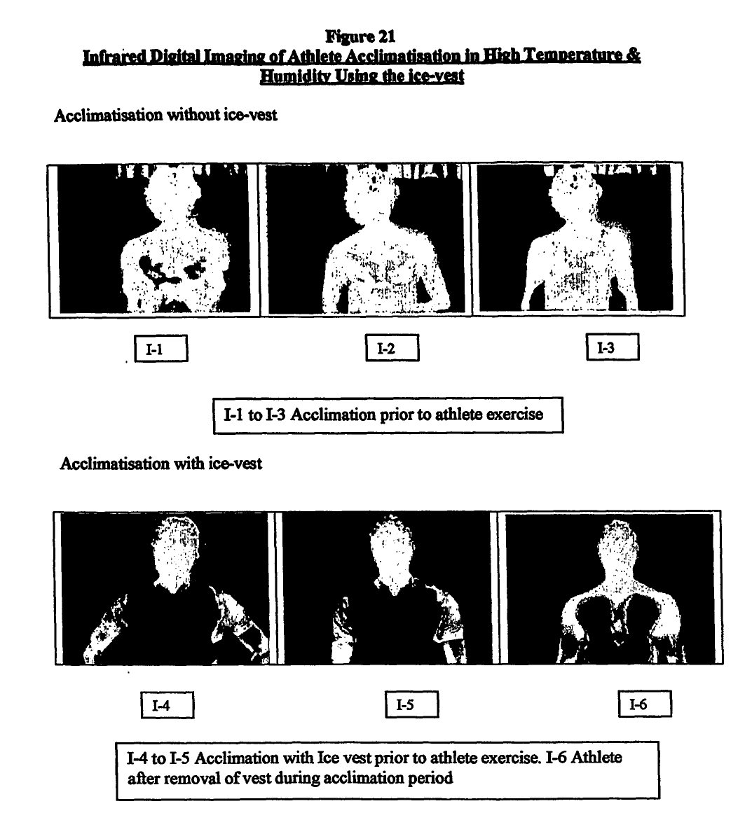

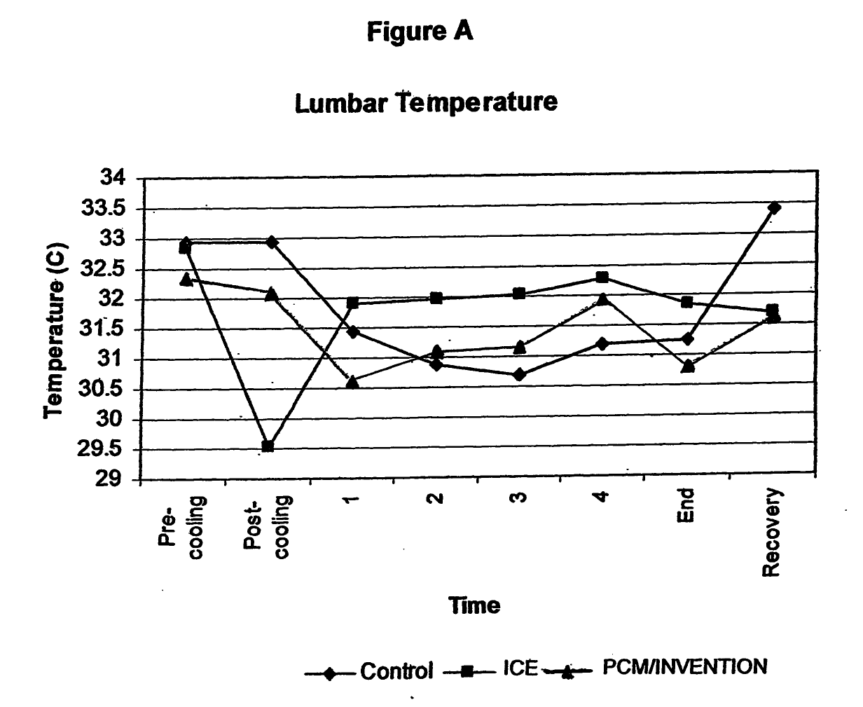

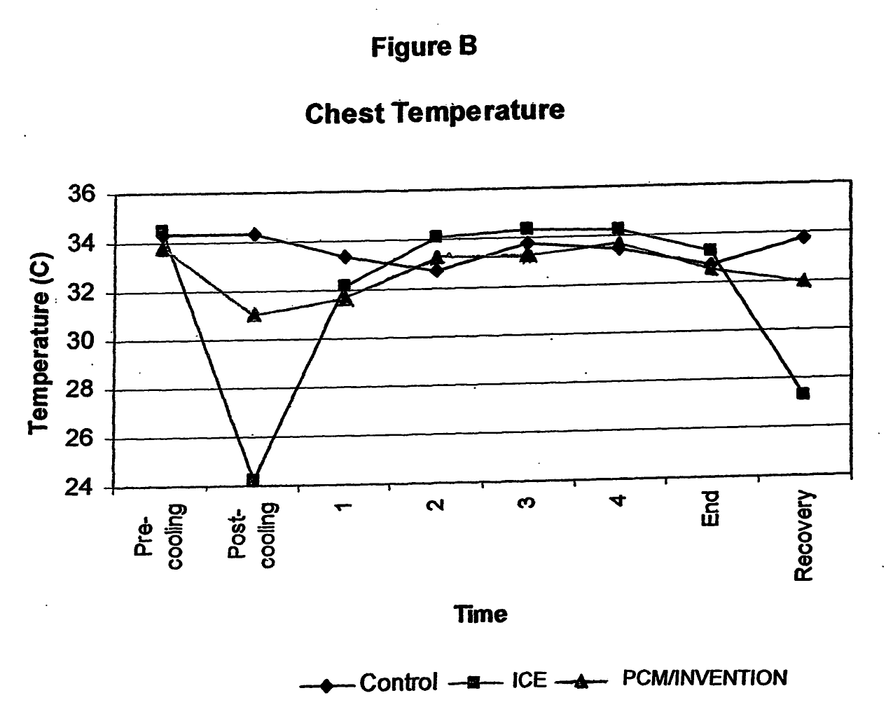

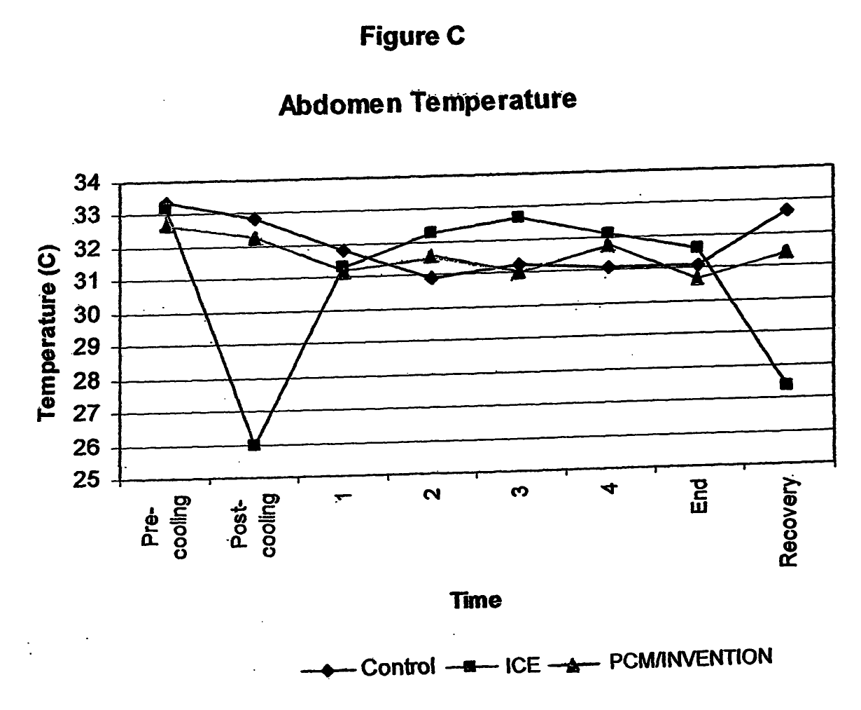

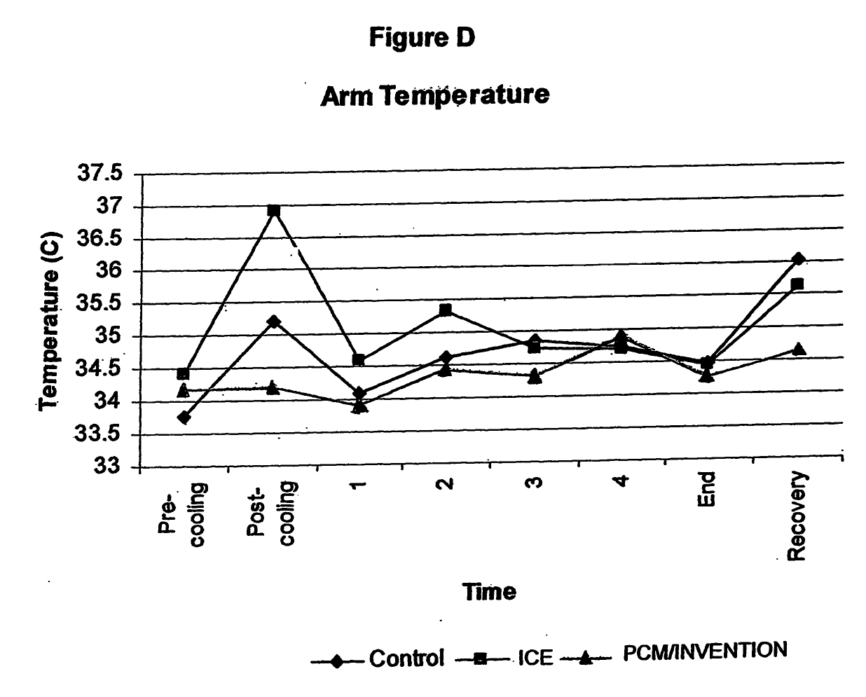

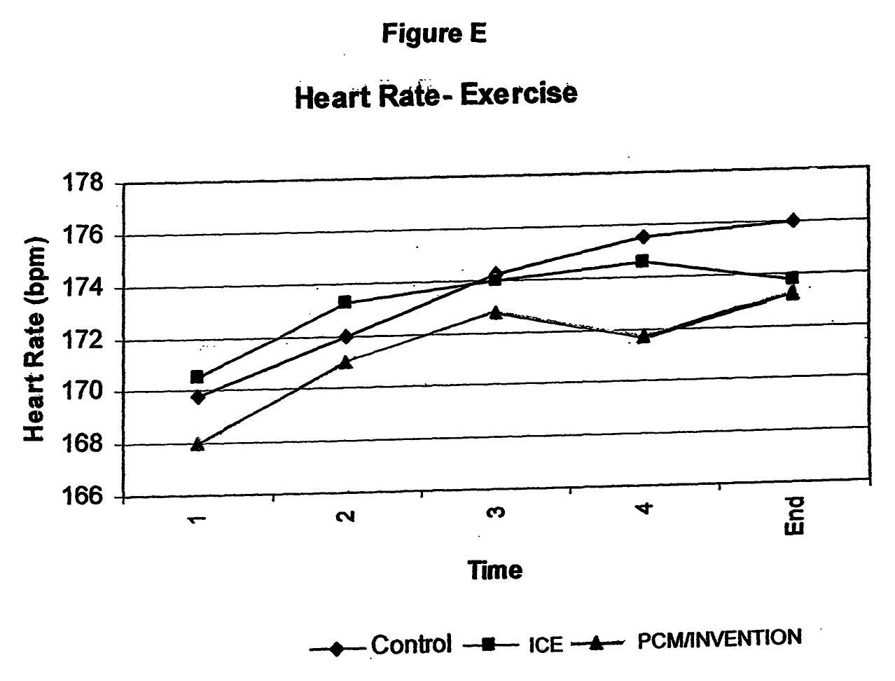

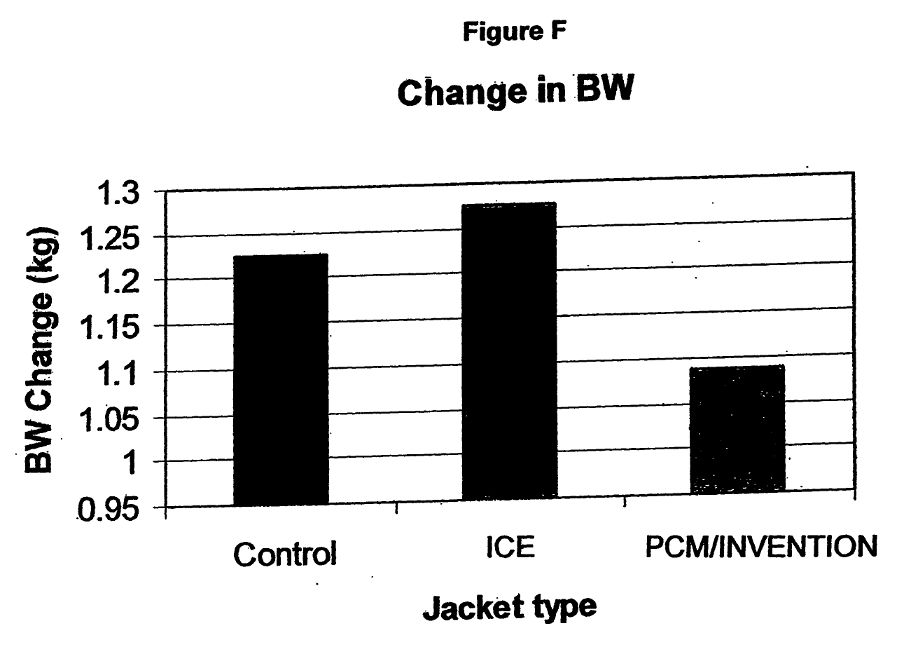

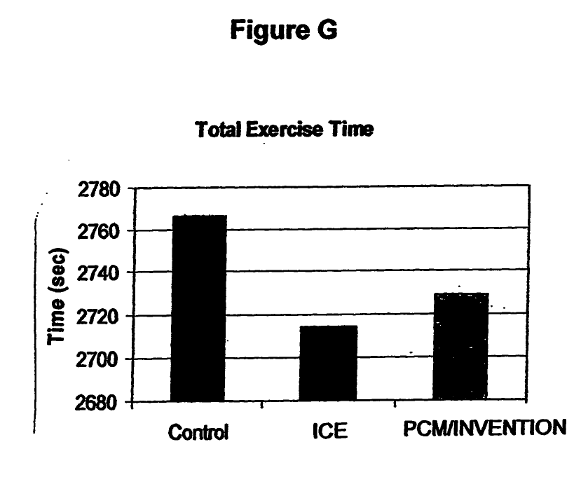

Infrared digital imaging was used to quantify each athlete's skin temperatures during the evaluation trials. FIGS. 20 and 21 show the athlete's skin temperatures for the jacket of the invention and the conventional ice vest respectively. Images are shown for the acclimatisation periods prior to exercise with and without the use of the cooling garments. Also included are images of each athlete after removal of the respective cooling garments.

[image]

The significance of the R1-R3 pictures is that they show how the athlete's body is acclimatising to the conditions within the environmental chamber. Initially (in shots—R1 and R2), he is quite hot throughout his upper chest/shoulder, neck and head area as these areas are red. After a time, he cools down (R3), as demonstrated by the colour change (from red to yellow, green or orange) in the eyes, nose and upper chest areas. These pictures show the body adjusting to the humid/hot conditions without the aid of a cooling garment.

The most significant thing to note in comparing the thermal imaging is the difference in the head temperatures after using the cooling garments.

The conventional ice-vest does not cool the head appreciably (the head still stays red) while the jacket of the invention cools the head. Even after the vest/jacket has been removed, R6 and I6, only the cooling jacket of the invention allows the athlete's head to remain cooled (R6). The athlete wearing the ice-vest still has a hot head after the jacket has been removed (I6).

Also, both sets of images show the importance of reconfiguring the jacket of the invention so that we could increase the amount of cooling medium onto the upper back and shoulders to deal with the higher thermal of the body in these areas.

The greater reduction in skin temperature achieved by the jacket of the invention is consistent with the greater reduction in athlete core body temperature shown in FIG. 7.

### EXAMPLE 2

This example describes PCM pouches useful in practice of the invention, design features of a variety of cooling garments incorporating the PCM pouches and typical applications of the garments.

Laminate Cooling Pouches

Each cooling garment includes two different sized PCM pouches. The vacuum packaging technique used required that the original pouch inter-seal dimensions were 110 mm×100 mm (size 1) and 70 mm×100 mm (size 2). However, in this example the PCM was a liquid under the prevailing conditions under which the pouch was filled. The PCM was then solidified by cooling and the pouch sealed. However, the pouches are made according to the principles explained in Example 1. The pouches are to be accommodated in 50 mm wide fabric pockets provided on the inside of the cooling garment. To achieve this the pouches are folded (along the 100 mm dimension) and secured in the folded configuration using adhesive tape. Ideally, suitably sized pouches would be made thereby avoiding the need to fold the pouches to fit into the pockets. The PCMs used were Rubitherm RT 2 and RT 20.

The laminate film used to form the pouches was RA463 (Cryovac) having a thickness of 68 μm.

In the table below are some dimensions related to the cooling packs.

| | Size 1 | Size 2 |
|---|---|---|
| RT 20 pouches | | |
| Average product volume (ml) when filled with RT 20 | 51 | 32 |
| Average product mass (g) when filled with RT 20 | 38 | 24 |
| Average pack thickness (cm) | 0.9-1.2 | 0.9-1.2 |
| Average surface area per pack (cm²) | 45.8 | 28.2 |
| RT 2 pouches | | |
| Average product volume (ml) when filled with RT 2 | 48 | 28 |
| Average product mass (g) when filled with RT 2 | 37 | 22 |
| Average pack thickness (cm) | 0.9-1.2 | 0.9-1.2 |
| Average surface area per pack (cm²) | 45.8 | 28.2 |

Vests (i.e. without sleeves) typically contain a minimum of 70 packs and a maximum of 106 packs and jackets contain a minimum of 106 packs and a maximum of 150 packs. Hoods contain 16 packs.

Most of the following design criteria addressed the issues of maximising the overall cooling effect, the duration of cooling sensation and increasing wearer comfort. Additional design features were also included even though they may not have functional significance.

Design Features

Jackets:
Logos stitched on collar
Yoke design introduced to increase loading in upper back and chest areas,
logos printed on collar
Waist draw strings for maximum body contact, zips in sleeves, thinner
PCM pouches. Logos printed on collar
Buckled belts
Zippers extending from sleeve underside from wrist to arm to facilitate
quick removal and putting on of garment and to ensure a close fit
Vests (no sleeves):
New design line pull garment close to body, underarm ribbing to reduce
pre-melting by warm air, no logos
Same as above
Buckled belts and logos on collars and back.
PCM pouches located along spine, upper back and chest areas.
Hoods:
Without straps
Velcro straps and loaded with additional 0.3 kg of RT 20.
Replaced Velcro with fabric tie straps, ribbing attached to front of hood
(around face) to provide a close fit.

### EXAMPLE 3

Early calculations of maximum amount of heat to be extracted from athletes may be used to direct the amount of PCM loading in the cooling garment. The table below shows the values of heat loss calculations and initial loading calculations for the PCM Rubitherm RT 20.

Estimates of Athlete Heat Losses

| Event | Heat loss/min | | Total Heat Loss |
|---|---|---|---|
| 4000 m cycling (individual) | 103.5 | kJ | 414 kJ |
| ˜4 min | | | |
| 42 km marathon | 80 | kJ | 10,400 kJ |
| ˜130 min | | | |
| At rest | 11.8 | kJ | 354 kJ (over 0.5 h) |
| During moderate exercise | 47 | kJ/min | 1,419 kJ (over 0.5 h) |

For a cooling garment to be useful for this range of activities it will have a minimum of 278 kJ and probably closer to 700-900 kJ (˜985 kJ) in 20-30 min.

| Heat Loss | Mass of RT 20 to Absorb Heat |
|---|---|
| 278 kJ (minimum) | 1.616 kg |
| 985 kJ (maximum) | 5.727 kg |

From this it can be seen that in principle 5.727 kg of PCM is required to accommodate the maximum heat loss likely to occur. In practice for comfort, manageability and transportability the garment will include an average of the required PCM amount for the minimum and maximum heat loss values, i.e. about 3.67 kg PCM RT 20.

### EXAMPLE 4

Several experiments were carried out on the duration of cooling required to completely activate whole garments after complete melting of PCM had occured of the type referred to in Example 2. Hoods (0.5 kg of PCM) require about 5.5 hours (RT 20 in fridge (about 4° C.) and RT 2 in freezer (about −18° C.)) when none of the inner pocket layer is exposed to the cold chamber. A jacket containing 3.97 kg of RT 2 requires 8 hours in a freezer (about −18° C.) before becoming totally charged and a vest including 3.41 kg of RT 20 needs about 8 hours in a fridge (about 4° C.) or overnight in an air-conditioned cool room (16° C.). The RT 20 jacket requires about 4 hours in a freezer (about −18° C.) to charge. This is in accordance with exposing the maximum amount of inner pocket surface area to the cold ambient temperature of a cold/cool chamber.

A certain amount of heat is required to warm the PCM to the phase change temperature for it to melt (Tm-melting temperature). The lower the storage temperature, a larger amount of heat will be required to change the PCM temperature.

The table below summarises that an additional 18 to 21% of additional heat may be loaded for body cooling when a garment containing ˜3.9 kg of RT 20 PCM is completely charged in fridge or freezer chambers (Ts denotes the storage temperature within the chamber).

| | | Heat Absorbed* in | | | |
|---|---|---|---|---|---|
| RT 20 mass Contained | Total Heat stored by RT | Warming from Tₛ to Tₘ | | | |
| in Garment (kg) | 20 | 2° C. | 5° C. | 15° C. | 20° C. |
| 3.899 | 670.6 kJ | 140 kJ | 119 kJ | 49 kJ | 14 kJ |

| | | | | |
|---|---|---|---|---|
| % total heat stored by RT 20 in Garment | 21 | 18 | 7 | 2 |

where:
RT 20 heat of fusion = 0.172 kJ/g, heat capacity (solid/liquid: dsc 10 kJ/min, value ± 10%) = 1.8/2.4 kJ/kg.K and Tₘ = 22° C.
*Heat absorbed in warming from Tₛ to Tₘ = mass of RT 20 × (Tₘ − Tₛ) × heat capacity (solid)

Subjective data that have been collected suggest that the garments are effective for about 40 minutes, that is from the time of donning the garment and then removing it after cooling sensation is no longer apparent. This effective cooling duration applied to a RT 2 vest/hood combination when worn in the sun (25-30° C.) during an extreme exercise period (typical rowing warm-up session). Other pre-cooling applications showed both PCM jackets (RT 20 and RT 2) were effective over the 30 minute pre-cooling periods and 30-5 minute post-exercise periods.

### EXAMPLE 5

The cooling jacket, as described in Example 1, was evaluated in a second set of trials at the Northern Territory Institute of Sport (Darwin, Australia) using four marathon runners performing a 10.4 km run. Experiments were conducted with athletes over 3 days (randomized trials: Monday, Wednesday, Friday) between 11 am and 2 pm where ambient conditions on the 400 m track were between 22-30° C. and 30-60% RH. Each athlete wore a conventional ice vest, a jacket-hood combination in accordance with the present invention as explained in Example 1 to confirm the comparative effectiveness and impact on performance of the cooling garments.

The four athletes were studied over a time period in which the periods were defined: initial rest time (first 30 minutes, pre-cooling), exercise period (10.4 km=26 laps of the 400 m track) and 30 minutes (recovery period, post-cooling). The garments were worn only during the pre-event and post-event periods.

Physiological responses such as skin surface temperatures (forearm, scapula, lumbar chest, abdomen), core body (rectal), lactate, heart rate, sweat loss, split times as well as thermal sensation and perceived exertion ratings were collected from each athlete. Skin temperature data was collected via infrared temperature gun and core body temperature data was collected using core body temperature pills and via rectal thermistor temperature probes. Lactate concentrations were measured by taking capillary (ear lobe) blood samples. Physiological measures were taken before and after pre-cooling, after each lap set and after the recovery period. The data was averaged over the 4 subjects to obtain the plots described below.

Key improvements and enhancements resulting from wearing the jacket/hood combination in accordance with the present invention are included in the following discussion.
Skin temperatures were lower at sites of contact between the skin and ice than the jacket/hood of the present invention. While skin temperatures were between 3 and 6° C. lower than when in contact with ice and 3.5 and 10° C. lower than the control, lumbar, chest and abdomen areas recovered after first lap set to about the same skin temperatures as for the invention jacket/hood-cooled areas (see Figure A-C—plots of lumbar, chest and abdomen skin temperatures).
Due to long sleeve design of jacket of the invention, arm temps were reduced by 1° C. compared to the control condition whereas the arm temperature was 1° C. warmer than the control condition when the ice garment was donned in pre-cooling (see Figure D plot of arm temperature). Note that the chilling due to contact with the ice jacket is significant initially for the lumbar temperature but is not advantagous during extreme exercise when the the lumbar temperature, following ice vest removal, was consistently higher.

Heart rates were consistently 2 bpm lower for the jacket/hood of the invention cooled subjects over the exercise period. Immediately after pre-cooling, both cooling garments (ice and present invention) counteracted the 13 bpm increase in heart rate experienced by subjects that were not cooled prior to exercise (control) (see Figure E plot of heart rate).
Body weight change from pre- to post exercise periods shows that the ice-cooling induces 4% more sweating whereas cooling using the jacket/hood of the invention reduces sweating by 11% (see Figure F plot of body weight).
The total time taken to complete the 26 laps was shorter from pre-cooling, 47 seconds versus control for ice and 35 seconds for PCM compared to control condition (see Figure G).

The jacket in accordance with the present invention has been shown in this example to reduce arm temperatures, heart rates, reduce sweating and times to complete exercise significantly.

### EXAMPLE 6

The cooling jacket, as described in Example 1, was modified to include a different product as premoulded semi-rod shapes of PCM (Rubitherm RT 2) of two different volumes (48 ml (37 g) and 28 ml (22 g)) were inserted into the laminate pouches and the final seal was made using a vacuum heat sealing unit. Adequate volume was left within the pouch to accommodate volume expansion (about 10% on phase change of the RT 2 product). When filled and re-melted to obtain a flat-shaped compartment, the total thickness of the PCM pouch was about 1.5 cm. Further modifications to the garment included a drawstring for the waist to enhance optimal contact between the garment and the skin and sleeves with zippers from the wrist to under the arm for easy arm access in donning and removing the garment.

This low melting temperature PCM jacket/hood configuration and it's vest counterpart with hood was evaluated in a preliminary set of trials at the Australian Institute of Sport (Canberra, Australia) using an elite cyclist performing a typical cycling warm-up (high exertion exercise). Experiments were conducted with the subject at rest and exercising in a heat tent (37° C.). The subject wore the RT 2 jacket/hood configuration for pre-cooling to investigate the effect of lowering the operating temperature of the PCM within the cooling jacket on core body temperature over pre-cooling periods and the warm up periods, its effectiveness and impact on performance. The subject wore the RT 2 vest/hood configuration for cooling during intervals within the exercise period.

The subject was studied over a time period in which the periods were defined: initial rest time (60 minutes, pre-cooling) and exercise period (typical cycling warm-up period). The jacket/hood was worn during the pre-cooling period (60 minutes) and the vest/hood was worn between minutes 6-15 and 21-30 during the cycling warm up.

Physiological responses such as core body (rectal) via rectal thermistor temperature probes and heart rate were collected from the athlete. Physiological measures were taken before and after pre-cooling (60 minutes in hot chamber), during the exercise period (cycling warm-up completed twice; first 6 minutes 100 W power, next 5 minutes at 200 W power and last 2 minutes at 250 W power) and after the exercise period.

The results are summarised in the following table.

| | Tc change/values | | | |
|---|---|---|---|---|
| | before and after | Tc change/values before | | Tc change/values before |
| Cooling | cool shower | and after rest in heat | | and after warm up in heat |
| Method | (30 minutes) | (60 minutes) | | (30 minutes) |
| Control | — | 37.1 to 37.1 | (no change) | 37.1 to 37.9 (0.8° C. increase) |
| 6° C. | — | 37.5 to 37.2 | (0.3° C. decrease) | 37.1 to 38.3 (1.2° C. increase) |
| garments*/ | | | | |
| hoods | | | | |
| Shower only | 37.6 to 37.2 (0.4° C. decrease) | 37 to 36.9 | (0.1° C. decrease) | 36.9 to 37.8 (0.9° C. increase) |
| 6° C. | — | 37.5 to 37.2 | (0.3° C. decrease) | 37.1 to 38.3 (1.2° C. increase) |
| garments*/ | | | | |
| hoods only | | | | |
| Shower, 6° C. | 37.5 to 37.3 (0.2° C. decrease) | 37.1 to 36.0 | (1.1° C. decrease) | 36.0 to 37.2 (1.2° C. increase) |
| garments*/ | | | | |
| hoods | | | | |
| Shower, 20° C. | 37.4 to 36.9 (0.5° C. decrease) | 36.7 to 35.9 | (0.8° C. decrease) | 35.8 to 37.6 (1.8° C. increase) |
| garments*/ | | | | |
| hoods | | | | |

*Jacket/hood combination was worn during rest in heat period and vest/hood combination was worn between minutes 6-15 and 21-30 during the warm up

Conclusion: In a 37° C. ambient temperature, when the cooling protocol of either jacket/hood combination is used for 1 hour after a 0.5 hour cool shower period, an overall reduction of ˜1.5° C. in core body temperature can be achieved prior to high exertion exercise (cycling warm up).

After the cycling warm up period, when the vest/hood combination is utilized during the exercise period, the core body temperatures under this elevated ambient temperature is at or below the starting temperature (at rest).

Key improvements and enhancements resulting from wearing the jacket/hood combination in accordance with the present invention are included in the following discussion.
A drop of 0.3° C. in core body temperature during the pre-exercise cooling period when using the RT 2 cooling jacket/hood for 60 minutes in a hot tent. The corresponding control condition maintains core body temperature at a constant value over the whole hour while the subject is sitting in the hot tent.

Further experiments where conducted where the pre-cooling period was followed by a shower treatment where the subject stood under a cool water shower (23-28° C.) for 30 minutes (the same effect may be achieved by use of a cool chamber or the equivalent). This was followed by the subject wearing a jacket of the type described in Example 1 with an accompanying RT 20 hood or a Jacket as described in Example 2 with an accompanying RT 2 hood.

Key improvements and enhancements resulting from wearing either of the jacket/hood combinations in accordance with the present invention are included in the following discussion.
An initial core body temperature drop between 0.2 and 0.5° C. is achieved after a 30 minute cool shower and this may be extended and reduced by a further 0.8° C. (RT 20 garment/hood) and by a further 1.1° C. (RT 2 garment/hood) after these cooling garments are used for 60 minutes following the shower. When either jacket was not worn in the heat tent following a shower, the core body temperature was reduced by just 0.1° C. after 60 minutes of sitting in the hot tent.

The significance of these results means that both 20° C. and 6° C. melting point PCM jacket/hood assemblies produced a 1.5° C. reduction in core body temperature prior to the exercise period. From the combination of cool shower (first cooling period) for 30 followed by 60 minutes of wearing either RT 2 or RT 20 hood/jacket combinations (second cooling period), core body temperature are lowered significantly prior to exercise.
After the intense exercise period, where the vests are employed intermittently during the exertion, the core body temperature after using either RT 2 or RT 20 configurations showed that after warm up, the core body temperatures are at the same values as when the subjects were at rest prior to being cooled by any method.

These jacket/hood and vest/hood combinations in accordance with the present invention have been shown in this example to reduce core body temperature significantly after a warm-up cycling period when used following a water immersion/cool shower for 30 minutes.

### EXAMPLE 7

The cooling jacket, as described in Example 1, was evaluated in a third set of trials in Ballarat at the University of Ballarat (Victoria, Australia) using teams of elite male cyclists performing a warm-up and competition in a warm room (˜34° C.) after a 20-30 minute cool shower (25-29° C.) followed by a 40 minute period sitting in a warm room (˜34° C.) wearing the cooling garment as described in Example 1.

Each cyclist wore a the RT 20 jacket-hood combination in accordance with the present invention as described in Example 1 to confirm and investigate the impact of lowering core body temperature on cycling time-trial performance, power output and performance times.

The physiological response of core body (rectal) and measures of performance time, time trial performance and power output (watts) of the cyclists were collected. The results are summarised in the following table.

Summary of Results of Ballarat Trials

| Physiological/ | Control | | Jacket/hood after |
|---|---|---|---|
| Performance Measure | (normal) | Jacket/hood alone | pre-cooling in cool shower |
| 1. Core body | Increase of 38 | Increase of 38 to 39.70° C. | Increase of 37.42 to 39.65° C. |
| temperature | to 39.75° C. | (increase of 1.7° C., but | (increase of 2.23° C., but core |
| measures taken before | (increase of | 0.05° C. cooler than | body temp is 0.58 lower than |
| and after a ˜40 min time | 1.75° C.) | control) | control and jacket cooled subject |
| trial using Ballarat “A” | | | before the time trial and between |
| Grade Cyclists | | | 0.05 and 1° C. lower than |
| | | | jacket/hood cooling alone and |
| | | | control respectively). |

Conclusion: In a 34° C. ambient temperature, when both forms of pre-cooling are used, i.e, cool
shower for 30minutes (or other means of cooling) followed by wearing the RT 20 jacket/hood
combination for 40 minutes, the cyclist has a significantly lower core body temperature to begin
competition (time trial event) and this reduction carries through to after the time trial.

| | | | |
|---|---|---|---|
| 2. Performance Time | ˜2297 seconds | ˜2282 seconds | ˜2256 seconds |
| (seconds) | time trial with | decrease in 15 seconds | decrease in 41 seconds in time |
| measures taken from 6 | no cooling | in time trial | trial |
| Ballarat “A” grade | | (0.15% decrease) | (0.42% decrease) |
| Cyclists | | | |

Conclusion: Performance times may be reduced by as little as 15 seconds (jacket/hood configuration
alone) and as much as 41 seconds (when 30 minute cool shower is conducted before jacket/hood
cooling).

| | | | |
|---|---|---|---|
| 3. Time vs Time Trial | Each quarter | Each quarter takes | Each quarter takes between 255 |
| Splits | (time trial | between 250 and 285 | and 265 seconds to complete |
| measures taken from | splits: 25%, | seconds to complete | |
| 6 Ballarat “A” grade | 50%, 75% and | | |
| Cyclists | 100%) takes | | |
| | between 280 | | |
| | and 265 | | |
| | seconds to | | |
| | complete | | |

Conclusion: Time Trial Splits (quarters) may be reduced by a consistent 10 seconds throughout the
time trial (compared to the control) when both cooling techniques are used in combination.

| | | | |
|---|---|---|---|
| 4. Effect on Power | Control˜ power | — | After Pre-Cooling ˜ power |
| Output | output is 329 W | | output is 338 W |
| measures taken from | | | An increase of 2.75% in average |
| 6 AIS male cyclists | | | 30 min time trial power output. |

Conclusion: When both pre-cooling techniques are used, an increase of 9 watts in power output
over the average 30 minute time trial is achieved, equating to a 2.75% power output increase.

Key improvements and enhancements resulting from wearing the jacket/hood combination in accordance with the present invention are included in the following discussion.
The core body temperature is reduced by 0.5° C. when both pre-cooling methods (cool shower and cooling garment) are employed after a ˜40 minute time trial.
The performance time is reduced by 15 seconds (0.15% decrease) when the jacket is used alone to cool the cyclist and reduced by a further 26 seconds to 41 seconds (0.42%) when both pre-cooling methods are used together.
In terms of time trial splits, the combination of cool shower and jacket/hood cooling reduces time trials by a constant 10 seconds during the whole time trial period as compared to the control condition time trials.
A power output increase of 2.75% in 30 minute time trial power is seen after pre-cooling.

1. An article of clothing comprising a phase change material which is a blend of at least two compounds and which has a melting point of from 10 to 30° C. and a melting temperature range of from 1 to 5° C., wherein the phase change material is provided in discrete pouches and wherein the pouches are fitted into regions which in use are likely to come into proximity with body sites of high heat dissipation.

2. An article of clothing according to claim 1, wherein the phase change material, has a melting point of from 10° C. to 25° C.

3. An article of clothing according to claim 1, wherein the phase change material has a melting point of about 22° C.

4. An article of clothing according to claim 1, wherein the phase change material has a melting point range of from 1 to 4° C.

5. An article of clothing according to claim 1, wherein the phase change material has a heat of fusion of 150 kJ/kg to 250 kJ/kg.

6. An article of clothing according to claim 1, wherein the phase change material undergoes less than 17% expansion on complete melting.

7. An article of clothing according to claim 1, wherein the phase change material is encapsulated by a laminate film, wherein the laminate film comprises an outer heat-sealing layer and an inner layer which is impermeable to the phase change material.

8. An article of clothing according to claim 7, wherein the laminate is a three-layer film comprising a layer which is impermeable to the phase change material interposed between heat sealing layers.

9. An article of clothing according to claim 7, wherein the overall thickness of the laminate is from 30 to 150 μm.

10. An article of clothing according to claim 7, wherein the laminate comprises a layer of (biaxially oriented) nylon interposed between layers of LLDPE.

11. An article of clothing according to claim 10, wherein the nylon layer is from 10 to 50 cm thick and the LLDPE layers from 50 to 100 μm thick.

12. An article of clothing according to claim 11, wherein the nylon layer is about 15 μm thick and each layer of LLDPE about 51 μm thick.

13. An article of clothing according to claim 1, wherein the phase change material is contained in a number of individual and slender pouches which enable a flexible article of clothing to be prepared.

14. An article of clothing according to claim 1, wherein each pouch has a heat exchange surface area to volume ratio of from 1.06 to 1.20.

15. An article of clothing according to claim 1, wherein the pouches are inserted into pockets within the article and sealed therein.

16. An article of clothing according to claim 1, wherein the material from which the article is made is lightweight and breathable, and shaped so that in use the pouches will be in close proximity to the wearer's skin.

17. An article of clothing according to claim 1, in the form of a jacket or vest, wherein the phase change material is concentrated to provide a high level of cooling to the chest, back and/or shoulder areas.

18. An article of clothing according to claim 1, in the form of a jacket or vest, wherein the pouches are provided in a rib-like arrangement across the front and back of the jacket or vest.

19. An article of clothing according to claim 1, wherein the pouches do not extend over flex points in the article of clothing.

20. An article of clothing according to claim 1, comprising inner and outer shells, the inner shell being adapted to receive pouches containing the phase change material.

21. An article of clothing according to claim 1, further comprising one or more suitably positioned fittings that allow a thermal exchange surface of the article to be brought into close contact with the wearer's body, or parts thereof.

22. An article of clothing according to claim 1, comprising pouches containing different types of phase change materials depending upon the location of the pouch within the article of clothing.

23. An article of clothing according to claim 22, wherein in areas of the article corresponding to regions of the body of high heat loss, the pouch(es) contain a phase change material having a relatively high heat of fusion and wherein in areas of the article corresponding to regions of the body is where heat loss is lower the pouch(es) contain a phase change material having a lower heat of fusion.

24. An article of clothing according to claim 1, further comprising one or more ice packs.

25. An article of clothing comprising a phase change material having a melting point of about 6° C. and a phase change material having a melting point of about 22° C., wherein each phase change material has a melting temperature range of from 1 to 5° C.

26. Use of an article of clothing for pre-event, intra-event, inter-event and/or post-event cooling of an athlete, wherein the article of clothing comprises a phase change material which is a blend of at least two compounds and which has a melting point of from 5 to 30° C. and a melting temperature range of from 1 to 5° C.

27. Use of an article of clothing to reduce the core body temperature of an individual such that after completing a warm-up or pre-event routine without the article of clothing the core body temperature of the individual is approximately the same, or even slightly lower, than that before cooling was initiated, wherein the article of clothing comprises a phase change material which is a blend of at least two compounds and which has a melting point of from 5 to 30° C. and a melting temperature range of from 1 to 5° C.

28. Use according to claim 27, wherein the desired overall reduction in core body temperature is achieved by using the article of clothing before, after or during application of some other method of cooling the body.

29. Use according to claim 28, wherein the other method of cooling the body is a cool or cold shower.

30. Use of an article of clothing according to claim 28 to provide a cooling helmet.

31. Use of an article of clothing according to claim 1, to provide a cooling garment to be worn under protective clothing.

32. Use of an article of clothing according to claim 1 in medicine where cooling of the body or a part thereof is beneficial.

33. Use according to claim 32 for the management and/or treatment of ectodermal dysplasia, multiple sclerosis or head trauma.

34-36. (canceled)
